(19) United States
(12) Patent Application Publication
(10) Pub. No.: US 2025/0003953 A1
(43) Pub. Date: Jan. 2, 2025

(54) Methods and Model Systems for Assessing Therapeutic Properties of Candidate Agents and Related Computer Readable Media and Systems

(71) Applicant: The Regents of the University of California, Oakland, CA (US)

(72) Inventors: Hani Goodarzi, San Francisco, CA (US); Johnny Yu, San Francisco, CA (US)

(21) Appl. No.: 18/580,417

(22) PCT Filed: Jul. 22, 2022
(86) PCT No.: PCT/US2022/038069

§ 371 Date: Jan. 18, 2024

Related U.S. Application Data
(60) Provisional application No. 63/225,209, filed on Jul. 23, 2021.

(51) Int. Cl. G01N 33/50; C12N 5/00; C12Q 1/6869; G16B 20/00; G16B 25/10

(52) U.S. Cl. CPC G01N 33/5023; C12N 5/0062; C12Q 1/6869; G16B 20/00; G16B 25/10; C12N 2503/02; C12N 2513/00

(57) ABSTRACT

Provided herein are in-vitro, in-vivo, and ex-vivo models systems, methods of creating such model systems, and methods of using such model systems for assessing one or more therapeutic properties of a candidate agent or identifying a new therapeutic target. Also provided are computer-readable media and systems that find use, e.g., in practicing the methods of the present disclosure.

### CROSS-REFERENCE TO RELATED APPLICATIONS

This application claims the benefit of U.S. Provisional Patent Application No. 63/225,209, filed Jul. 23, 2021, which application is incorporated herein by reference in its entirety.

### STATEMENT OF GOVERNMENT SUPPORT

This invention was made with government support under grant no. R00 CA194077 awarded by The National Institutes of Health. The government has certain rights in the invention.

### BACKGROUND

The recent phenomenon of rising drug discovery costs and diminishing returns in therapeutic molecule discovery can be attributed to the foundations of drug discovery science. Specifically, high-throughput screening (HTS) has been and remains the gold standard for new molecule discovery. However, a key limitation of HTS has been its contrived nature—it is only possible in in vitro biochemical or cell-based assays. In vitro biochemical or cell-based assays followed by preclinical in vivo studies fail to provide sufficient pharmacological and toxicity data or reliable predictive capacity for predicting a therapeutic drug candidate performance in vivo making the drug development process costly and inefficient. The present disclosure provides in-vivo and ex-vivo models systems and methods of creating such systems for performing scalable HTS screening.

### SUMMARY

Provided are a balanced cell count culture and method of creating the same, methods for assessing one or more therapeutic properties of a candidate agent. The methods comprise growing a heterogeneous pool of cells of different cell types in three dimensions, treating the three dimensional pool with the small molecule compound, and dissociating cells of the treated three dimensional pool into a single-cell suspension with equal representation of cell types suitable for single-cell RNA sequencing. The methods further comprise performing single cell ribonucleic acid (RNA) sequencing on the dissociated single cells and dissociated single cells from a control three dimensional pool not treated with the small molecule compound, deconvoluting the data from the single cell RNA sequencing into single cell transcriptomes categorized by treatment and cell type, and assessing one or more therapeutic properties of the small molecule compound based on the categorized single cell transcriptomes. Also provided are computer-readable media and systems that find use, e.g., in practicing the methods of the present disclosure.

### BRIEF DESCRIPTION OF THE FIGURES

FIG. 1A-1G demonstrates the comparison of cell composition between non-GENEVA and a GENEVA cell pools. FIG. 1A demonstrates distribution of cell representation from a non-GENEVA cell pool (Pool 1) based on single cell RNA sequencing harvest. Bars indicate the number of cells in the scRNAseq dataset for each cell type. FIG. 1B shows the dataset from FIG. 1A plotted in two-dimensional transcriptome space using a UMAP clustering visualization algorithm. FIG. 1C shows distribution of cell representation upon single-cell RNA sequencing harvest from a GENEVA cell pool (Pool 2) allowing for accurate capture of each cell line within the dataset. FIG. 1D shows single cell RNA sequencing data plotted as UMAP plots for GENEVA pool 2. FIG. 1E shows distribution of cell representation upon single-cell RNA sequencing harvest from a GENEVA cell pool (Pool 3) allowing for accurate capture of each cell line within the dataset. FIG. 1F shows single cell RNA sequencing data plotted as UMAP plots for GENEVA pool 3. FIG. 1G shows extrapolation of Pools 1-3. The total number of cells required for single-cell RNA sequencing is significantly higher in Pool 1 compared to Pool 2 and Pool 3.

FIG. 2A-F demonstrates the utilization of GENEVA in multiple in-vivo and ex-vivo model systems. FIG. 2A demonstrates a GENEVA pool grown as organoids ex-vivo with four different human PDX tumors as input, treated with several doses of ARS1620 (0.4 uM, 1.6 uM, 25.0 uM) or DMSO (vehicle). FIG. 2A is plotted in two-dimensional transcriptome space using a UMAP clustering visualization algorithm. FIG. 2B shows the dataset from FIG. 2A as a table categorized by the drug treatment conditions and genotype (PDX) of origin. Cells within the table are the cell counts obtained by single-cell RNA sequencing for each category. FIG. 2C demonstrates a GENEVA pool grown as a flank xenograft in-vivo with four different human PDX tumors as input, treated with either ARS1620 (100 mg/kg) or DMSO (vehicle). FIG. 2C is plotted in two-dimensional transcriptome space using a UMAP clustering visualization algorithm. FIG. 2D shows the dataset from FIG. 2C as a table categorized by the drug treatment conditions and genotype (PDX) of origin. Cells within the table are the cell counts obtained by single-cell RNA sequencing for each category. FIG. 2E demonstrates a GENEVA pool grown as a flank xenograft in-vivo with eight different human cancer cell lines as input, treated with either ARS1620 (100 mg/kg) or DMSO (vehicle). FIG. 2E is plotted in two-dimensional transcriptome space using a UMAP clustering visualization algorithm. FIG. 2F shows the dataset from FIG. 2E as a table categorized by the drug treatment conditions and genotype (PDX) of origin. Cells within the table are the cell counts obtained by single-cell RNA sequencing for each category.

FIG. 3A-E demonstrates the utilization of GENEVA for discovery of relative phenotype to drug compound, genetic drivers, and IC50 curve reconstruction. FIG. 3A demonstrates relative sensitivity of individual cell types calculated from pre/post drug treatment relative cell counts from GENEVA pools treated with Vemurafinib or ARS1620. The most sensitive cell lines in Vemurafinib treated pools are BRAF.V600E mutant harboring. The most sensitive cell lines ARS1620 treated pools are KRAS.G12C mutant harboring. FIG. 3B demonstrates discovery of causal driver mutations responsible for changes in relative drug sensitivity in a GENEVA pool using lasso regression models. BRAF.V600E is predicted by the lasso algorithm as the responsible mutation causing drug sensitivity to Vemurafinib. KRAS.G12C is predicted by the lasso algorithm as the responsible mutation causing drug sensitivity to ARS1620. FIG. 3C demonstrates reconstruction of IC50 curves from GENEVA cell pool data after treatment with and without ARS1620 with cell counts fitted to a scaled measure of relative percent survival and IC50 logistic regression curves interpolated. IC50 curves are constructed from individual cell lines and non-KRAS.G12C cell lines show significantly greater survival to ARS1620 than KRAS.G12C cell lines. FIG. 3D demonstrates relative drug sensitivity measurements from different cell lines in a GENEVA pool thereby recapitulating discovery of KRAS.G12C as the sensitizing mutational target for ARS1620. FIG. 3E demonstrates calculation of Cell Cycle Inhibition Rates from GENEVA performed in PDX grown as pooled organoids. This reconstruction method recapitulates KRAS.G12C-specific drug sensitivity to ARS-1620 treatment by an alternative method to cell counting using cycle state inference as a measurement of phenotype.

FIG. 4A-D demonstrates the utilization of GENEVA for prediction of combination therapy and drug resistance mechanisms. FIG. 4A demonstrates GENEVA discovers upregulation of several drug resistance targets indicating cellular survival mechanisms in a GENEVA pool treated with ARS1620 in a KRAS.G12C specific fashion. FIG. 4B demonstrates validation of predicted drug targets by dosing drug targets in combination with i) three ARS1620 inhibitors and ii) compounds targeting a specific drug resistance pathway. Bliss drug synergy is plotted and several compounds show significant drug synergy with multiple KRAS.G12C inhibitors. FIG. 4C demonstrates relative tumor volume from in-vivo mouse studies of ARS1620 and INK128 in a multi-arm combination therapy using H1373 KRAS.G12C mutant line (n=4-5 mice per condition). FIG. 4D demonstrates INK128 and ARS1620 synergistically reduce tumor growth in-vivo compared to a null model of INK128 and ARS1620 independence or no drug synergy.

FIG. 5A-C demonstrates the utilization of GENEVA for prediction of an in-vivo specific mechanism of drug resistance via the endothelial-mesenchymal transition (EMT) pathway. FIG. 5A demonstrates that in a paired in vivo and in vitro GENEVA experiment of ARS1620 treatment of KRAS.G12C cell lines in a cell pool, the EMT geneset was upregulated post-drug treatment in vivo but not in vitro. FIG. 5B demonstrates relative tumor volume from in-vivo mouse studies of ARS1620 and Galunisertib, an EMT inhibitor, in a multi-arm combination therapy using H1373 KRAS.G12C mutant line (n=4-5 mice per condition). FIG. 5C demonstrates Galunisertib and ARS1620 synergistically reduce tumor growth in-vivo compared to a null model of Galunisertib and ARS1620 independence or no drug synergy.

FIG. 6A-E demonstrates the utilization of GENEVA for discovery of molecular mechanism of action of the compound on mitochondrial genes. FIG. 6A plots aggregated gene expression across KRAS.G12C lines in GENEVA pools of mitochondrially encoded and genomically encoded mitochondrially-targeted transcripts compared to gene expression of non-mitochondrial gene transcripts after ARS1620 treatment. Mitochondrially encoded genes and genomically encoded mitochondrial resident genes are significantly downregulated in cells surviving ARS1620 treatment. FIG. 6B plots gene expression of mito-encoded transcripts after ARS1620 treatment for each individual KRAS.G12C cell line in the GENEVA cell pool. FIG. 6C demonstrates generation and profiling of a long term ARS1620 tolerant cell line (30 day treatment, 10 uM) from H2030 (KRAS.G12C). Assay of mitochondrial content using fluorescent mitochondrial stain (Mitotracker Deep Red FM) between the H2030 drug-persistent cell line and the original parental cell line shows a decrease of mitochondrial content after long-term drug treatment with ARS1620. FIG. 6D demonstrates that the KRAS.G12C inhibitor AMG510 increases mitochondrial respiration and electron transport chain activity as novel lethality mechanisms of KRAS.G12C inhibition using a seahorse assay measuring oxygen consumption of H2030 (KRAS.G12C) cells at (2 h) after AMG510 treatment. FIG. 6E demonstrates that subpopulation structure of KRAS.G12C cell lines show selection for cell types with low numbers of mitochondrial reads post-treatment with ARS1620.

FIG. 7A-G demonstrates the utilization of GENEVA for discovery of molecular mechanism of action of the compound on ferroptosis genes. FIG. 7A plots a volcano plot showing Z-score differences aggregated across multiple G12C lines from GENEVA pools drugged with ARS1620 and demonstrates a shared upregulation of anti-ferroptotic genes. FIG. 7B plots gene expression of anti-ferroptotic genes for each cell line in response to increasing ARS1620 dosage. FIG. 7C utilizes experimental investigation of ferroptosis using a fluorescent lipid peroxidation sensor to demonstrate dose-response of cells of lipid peroxidation to ARS1620 dosage (48 h). FIGS. 7D, E, F demonstrates survival and lipid peroxidation kinetics across known ferroptotic agents Altretamine in FIG. 7D, Erastin in FIG. 7E as compared to ARS1620 in FIG. 7F. Lipid peroxidation and survival kinetics cross over around IC50 in all compounds indicating ARS1620 performs as a ferroptosis inducing agent. FIG. 7G demonstrates multiple KRAS inhibitors induce lipid peroxidation in a KRAS.G12C cell line H2030 specifically but not as much in KRAS.G12V cell line H441.

FIG. 8A-D demonstrates GENEVA testing of combination therapies incorporating multiple co-dosed compounds in cell pools in-vivo. FIG. 8A demonstrates a GENEVA combination therapy study using CLX pools in KRAS.G12 mutant lines categorized by cell line of origin and drug treatment conditions, plotted in two-dimensional transcriptome space using a UMAP clustering visualization algorithm. Treatment conditions include Antimycin, ARS1620, Galunisertib, INK128, DMSO, ARS1620+Antimycin, ARS1620+Galunisertib, ARS1620+INK128. FIG. 8B utilizes GENEVA data from FIG. 8A for synergy calculations performed on cell cycle states in each drug condition singly and in combination estimated for G12C lines across drug conditions. FIGS. 8C-D demonstrate identification of genes driving synergistic drug effect of Galunisertib (FIG. 8.C) and INK128 (FIG. 8.D) in combination with ARS1620 using a multifactorial linear model estimating gene-level synergy reveal mitochondrial transcripts to be driving synergistic drug effect.

FIG. 9A-B demonstrate genetic demultiplexing improvement algorithm and identification of novel genotypes of patients that would respond to ARS1620 by GENEVA method. FIG. 9A demonstrates the improvement in cell assignment confidence by genetic demultiplexing denoising algorithm comparison against standard method of percent representation from a Totalseq labelled single-cell RNA sequencing dataset. Higher confidence metrics are noted in the dotted line, or Noise Corrected Algorithm results. FIG. 9B plots a GENEVA pool drugged with and without ARS1620 demonstrated sensitivity of EML4-ALK as the most drug sensitive tumor type where each bar is the relative survival of that genotype under ARS1620 treatment or vehicle.

### DETAILED DESCRIPTION

Before the methods, computer readable media and systems of the present disclosure are described in greater detail, it is to be understood that the methods, computer readable media and systems are not limited to particular embodiments described, as such may, of course, vary. It is also to be understood that the terminology used herein is for the purpose of describing particular embodiments only, and is not intended to be limiting, since the scope of the methods, computer readable media and systems will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, computer readable media and systems. The upper and lower limits of these smaller ranges may independently be included in the smaller ranges and are also encompassed within the methods, computer readable media and systems, subject to any specifically excluded limit in the stated range. Where the stated range includes one or both of the limits, ranges excluding either or both of those included limits are also included in the methods, computer readable media and systems.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, computer readable media and systems belong. Although any methods, computer readable media and systems similar or equivalent to those described herein can also be used in the practice or testing of the methods, computer readable media and systems, representative illustrative methods, computer readable media and systems are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, computer readable media and systems are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, computer readable media and systems, which are, for clarity, described in the context of separate embodiments, may also be provided in combination in a single embodiment. Conversely, various features of the methods, computer readable media and systems, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, computer readable media and systems and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods

In one aspect, the present disclosure provides a balanced cell count culture and methods of creating the balanced cell count culture. In one aspect, the present disclosure provides methods for assessing one or more therapeutic properties of a candidate agent, e.g., a small molecule compound. The methods comprise growing a heterogeneous pool of cells of different cell types in three dimensions, treating the three dimensional pool with the small molecule compound, and dissociating cells of the treated three dimensional pool into single cells in a way that allows for equal representation of cells from different cell types. The methods further comprise performing single cell ribonucleic acid (RNA) sequencing on the dissociated single cells and dissociated single cells from a control three dimensional pool not treated with the small molecule compound, deconvoluting the data from the single cell RNA sequencing into single cell transcriptomes categorized by treatment and cell type, and assessing one or more therapeutic properties of the small molecule compound based on the categorized single cell transcriptomes.

The present methods address this by mixing the cells together, drugging them together, and then reading out the post-drug treatment cell lines using single cell-RNA sequencing. In this way, by reducing the unit of observation to a single cell and by mixing/multiplexing cell lines together, the present methods enable assaying of a large number of phenotypic/genotypic different cell lines against many small molecules. The resulting single cell RNA-sequencing data is analyzed using different models to discover biological targets, effective synergistic combination therapy targets, disease subtype stratification, and/or the like.

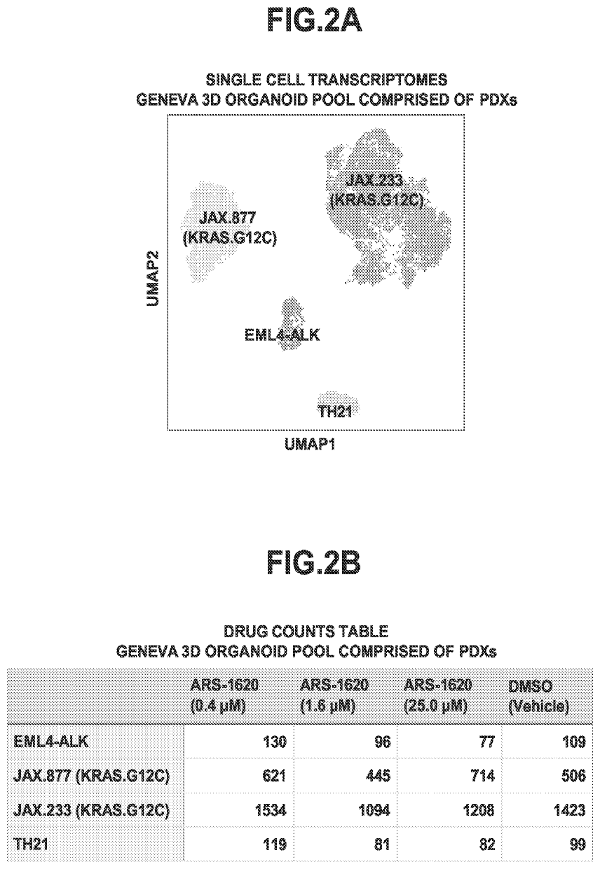

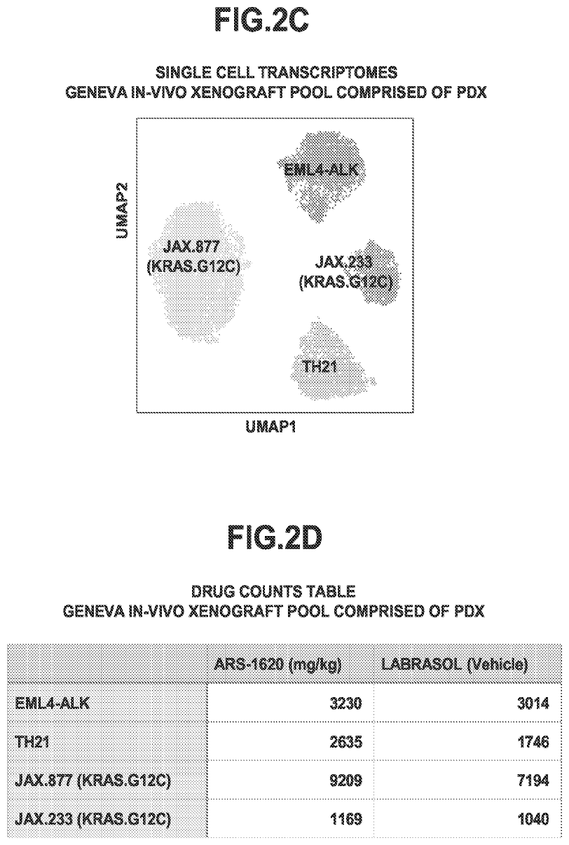

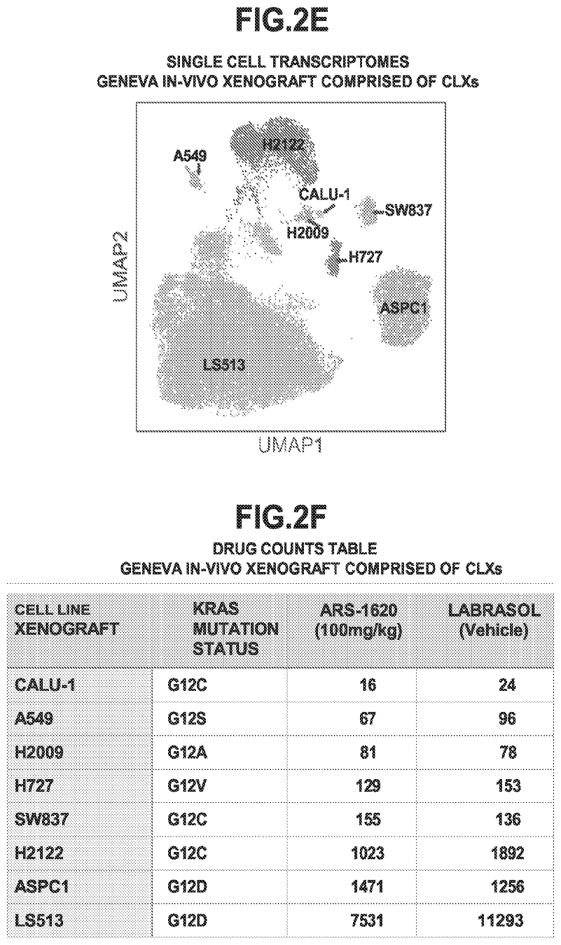

Embodiments of the methods of the present disclosure is provided in FIG. 2 as single-cell RNA sequencing results. In this example, a large panel of cell types from different patients, organ systems, and/or disease models/subtypes are mixed together to create a pool. The pool is then grown in three dimensions in vivo (e.g., producing a xenograft in an animal model, e.g., a mouse) or ex vivo (e.g., producing an organoid). The three-dimensional pool is then treated with an investigational small molecule compound of interest under conditions suitable for the compound to act on members (cells) of the three dimensional pool. The drug delivery method will vary depending upon the type of three-dimensional pool, e.g., systemic injection or the like when the three dimensional pool is an in vivo xenograft, etc. Next, the treated three-dimensional pool is harvested and dissociated into single cells which are then subjected to single cell RNA-sequencing. Phenotypic changes are noted by counting the number of individual viable cells for each cell types and by comparing it to the number of viable cells for each cell type from an identical three-dimensional pool that is not treated with the investigational small molecule compound of interest. The single cell sequencing data is then subjected to modeling and/or transcriptome analyses to assess one or more therapeutic properties of the small molecule compound, non-limiting examples of which include mechanism of action (MOA), combination therapy (drugs that would be effective as clinical combination therapies), and subtype stratification (efficacy in different patient groups or subtypes).

As summarized above, the methods of the present disclosure comprise growing a pool of cells of different cell types in three dimensions. In certain embodiments, the pool of cells of different cell types comprises 1000 or fewer, 500 or fewer, 250 or fewer, or 100 or fewer, but 2 or more, 5 or more, 10 or more (e.g., from 10 to 50), 20 or more, 30 or more, 40 or more, or 50 or more different cell types.

The cells of different cell types may be selected from any cell types of interest, which cell types may vary depending upon the particular small molecule compound of interest, the one or more therapeutic properties of the small molecule to be assessed, and/or the like. According to some embodiments, the pool of cells of different cell types comprises primary cells obtained from a patient, cells from an organ system, cells from a disease model, or any combination thereof.

Cells obtained from a patient may include, but are not limited to, cells from biopsy tissue obtained from a patient. Biopsy tissues may be obtained from healthy or diseased tissues, including e.g., cancer tissues. Depending on the type of cancer and/or the type of biopsy performed the cells may be from a solid tissue biopsy or a liquid biopsy. In some instances, the cells may be prepared from a surgical biopsy. Any convenient and appropriate technique for surgical biopsy may be utilized for collection of cells to be employed in the methods described herein including but not limited to, e.g., excisional biopsy, incisional biopsy, wire localization biopsy, and the like. In some instances, a surgical biopsy may be obtained as a part of a surgical procedure which has a primary purpose other than obtaining the sample, e.g., including but not limited to tumor resection, mastectomy, lymph node surgery, axillary lymph node dissection, sentinel lymph node surgery, and the like.

Various other biopsy techniques may be employed to obtain biopsy tissue, in turn to obtain cells to be employed in the methods of the present disclosure. As a non-limiting example, a sample may be obtained by a needle biopsy. Any convenient and appropriate technique for needle biopsy may be utilized for collection of a sample including but not limited to, e.g., fine needle aspiration (FNA), core needle biopsy, stereotactic core biopsy, vacuum assisted biopsy, and the like.

Cells from an organ system may include, but are not limited to, cells from on organ system selected from skin, brain, heart, kidney, liver, stomach, large intestine, lungs, and/or the like. According to some embodiments, cells from an organ system may include cells from on organ system selected from adrenal glands, anus, appendix, bladder (urinary), bone, bone marrow, brain, bronchi, diaphragm, ears, esophagus, eye, fallopian tube, gallbladder, genitals, heart, hypothalamus, joints, kidney, large intestine, larynx, liver, lung, lymph node, mammary gland, mesentery, mouth, nasal cavity, nose, ovaries, pancreas, pineal gland, parathyroid gland, pharynx, pituitary gland, prostate, rectum, salivary gland, skeletal muscle, smooth muscle, skin, small intestine, spinal cord, spleen, stomach, teeth, thymus gland, thyroid, trachea, tongue, ureter, urethra, ligament, tendon, hair, vestibular system, placenta, testes, vas deferens, seminal vesicles, bulbourethral glands, parathyroid gland, thoracic duct, arteries, veins, capillaries, lymphatic vessels, tonsils, neurons, subcutaneous tissue, olfactory epithelium (nose), cerebellum, and any combination thereof.

Cells from a disease model may include, but are not limited to, cells that model a disease selected from cancer (e.g., cells from one or more different cancer cell lines), cardiovascular disease, cerebrovascular disease (e.g., stroke, transient ischemic attack, subarachnoid hemorrhage, vascular dementia, etc.), respiratory disease, infectious disease, neurodegenerative disease, dementia, Alzheimer's disease, diabetes, kidney disease, liver disease (e.g., cirrhosis, nonalcoholic fatty liver disease (NAFLD), Hepatitis A, Hepatitis B, Hepatitis C, and/or the like), and any combination thereof.

According to some embodiments, the cells of different cell types comprise cells from one or more cancer cell lines. By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a hematological malignancy (e.g., a leukemia cell, a lymphoma cell, a myeloma cell, etc.), a primary tumor, a metastatic tumor, and the like.

When the cells of different cell types comprise cells from one or more cancer cell lines, the one or more cancer cell lines may be from a cancer independently selected from squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bile duct cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like. In certain embodiments, the one or more cancer cell lines may be from a cancer independently selected from a solid tumor, recurrent glioblastoma multiforme (GBM), non-small cell lung cancer, metastatic melanoma, melanoma, peritoneal cancer, epithelial ovarian cancer, glioblastoma multiforme (GBM), metastatic colorectal cancer, colorectal cancer, pancreatic ductal adenocarcinoma, squamous cell carcinoma, esophageal cancer, gastric cancer, neuroblastoma, fallopian tube cancer, bladder cancer, metastatic breast cancer, pancreatic cancer, soft tissue sarcoma, recurrent head and neck cancer squamous cell carcinoma, head and neck cancer, anaplastic astrocytoma, malignant pleural mesothelioma, breast cancer, squamous non-small cell lung cancer, rhabdomyosarcoma, metastatic renal cell carcinoma, basal cell carcinoma (basal cell epithelioma), and gliosarcoma. In certain embodiments, the one or more cancer cell lines may be from a cancer independently selected from melanoma, Hodgkin lymphoma, renal cell carcinoma (RCC), bladder cancer, non-small cell lung cancer (NSCLC), and head and neck squamous cell carcinoma (HNSCC). According to some embodiments, the cells of different cell types comprise cells from one or more cancer cell lines described in the Broad Institute Cancer Cell Line Encyclopedia (CCLE) available at portals.broadinstitute.org/ccle.

According to some embodiments, the cells of different cell types comprise cells from one or more different types of stem cells. Non-limiting examples of stem cells which may be included among the cells of different cell types include embryonic stem (ES) cells, adult stem cells, induced pluripotent stem cells (iPSCs), hematopoietic stem cells (HSCs), mesenchymal stem cells (MSCs), neural stem cells (NSCs), and any combination thereof.

As summarized above, the methods of the present disclosure comprise growing the pool of cells of different cell types in three dimensions. In certain embodiments, the pool of cells of different cell types is grown in three dimensions at least partially in vivo. According to one non-limiting example, growing the pool in three dimensions comprises producing a xenograft from the pool. As used herein, a “xenograft” is a tissue (including cell graft, e.g., cell line graft) from one species transplanted to a recipient of a different species. In certain embodiments, the donor species is human and the recipient animal is a mouse, rat, pig, or the like. The recipient animal may be immunodeficient (e.g., athymic nude mice, scid/scid mice, non-obese (NOD)-scid mice, recombination-activating gene 2 (Rag2)-knockout mice, etc.). When the recipient animal is a rodent (e.g., mouse or rat), the producing the xenograft may comprise parenteral injection of the pool of cells of different cell types into the recipient rodent, e.g., by tail vein injection. According to some embodiments, the xenograft is a cell line-derived xenograft (CDX), e.g., a xenograft comprising cells from one or more (e.g., two or more, three or more, four or more, five or more, 10 or more, or 25 or more) different cell lines, non-limiting examples of which include tumor cell lines. In certain embodiments, the xenograft is a patient-derived xenograft (PDX), e.g., a xenograft comprising primary cells (e.g., primary tumor cells) from one or more different patients, e.g., two or more, three or more, four or more, five or more, 10 or more, or 25 or more different patients. The primary cells may be obtained in some instance via a biopsy as described elsewhere herein.

In certain embodiments, the pool of cells of different cell types is grown in three dimensions at least partially ex vivo. The term “ex vivo” is used to refer to handling, experimentation and/or measurements done in or on samples (e.g., tissue or cells, etc.) obtained from an organism, which handling, experimentation and/or measurements are done in an environment external to the organism. Thus, the term “ex vivo manipulation” as applied to cells refers to any handling of the cells outside of an organism, including but not limited to culturing the cells, making one or more genetic modifications to the cells and/or exposing the cells to one or more agents. Accordingly, ex vivo manipulation may be used herein to refer to treatment of cells that is performed outside of an animal, e.g., after such cells are obtained from an animal or organ thereof. In contrast to “ex vivo”, the term “in vivo”, as used herein, refer to cells that are within an animal, e.g., rodent (e.g., mouse or rat), pig, etc.

In certain embodiments, the pool of cells of different cell types is grown in three dimensions at least partially in vitro. According to some embodiments, the pool of cells of different cell types grown in three dimensions is grown in vitro into an organoid. By “organoid” is meant a three-dimensional (3D) multicellular in vitro or ex vivo tissue construct that may mimic a corresponding in vivo organ. Organoids may be created through various types of available 3D cell culture systems, including but not limited to, 3D bioprinted scaffolds, organ-on-chip, microfluidics-based 3D cell culture models, and the like. According to some embodiments, the pool of cells of different cell types grown in three dimensions is grown in vitro into a spheroid. Organoids can be established for an increasing variety of organs, including but not limited to gut, stomach, kidney, liver, pancreas, mammary glands, prostate, upper and lower airways, thyroid, retina and brain-either from tissue-resident adult stem cells (ASCs), directly sourced from biopsy samples, or from pluripotent stem cells (PSCs), such as embryonic stem cells (ESCs) or induced PSCs (iPSCs). In certain embodiments, the pool of cells of different cell types grown in three dimensions is grown into a tissue-derived organoid, e.g., from one or more (e.g., two or more) different biopsy samples. Approaches for producing stem cell-derived and tissue-derived organoids are known and described, e.g., in Hofer & Lutolf (2021) Nature Reviews Materials 6:402-420.

Once the pool of cells of different cell types is grown in three dimensions, the three dimensional pool is treated with a small molecule compound. By “small molecule” compound is meant a compound (e.g., an organic compound) having a molecular weight of 1000 atomic mass units (amu) or less. In some embodiments, the small molecule is 900 amu or less, 750 amu or less, 500 amu or less, 400 amu or less, 300 amu or less, or 200 amu or less. In certain aspects, the small molecule is not made of repeating molecular units such as are present in a polymer. According to some embodiments, the small molecule compound is a known therapeutic agent. By “therapeutic agent” or “drug” is meant a physiologically or pharmacologically active substance that can produce a desired biological effect in a targeted site in an animal, such as a mammal or in a human. The therapeutic agent may be any inorganic or organic compound. A therapeutic agent may decrease, suppress, attenuate, diminish, arrest, or stabilize the development or progression of disease, disorder, or cell growth in an animal such as a mammal or human. In some embodiments, the small molecule compound is one approved by the United States Food and Drug Administration (FDA) and/or the European Medicines Agency (EMA) for use as a therapeutic agent in treating one or more diseases including but not limited to any of the diseases described elsewhere herein, e.g., cancer, cardiovascular disease, cerebrovascular disease, respiratory disease, infectious disease, neurodegenerative disease, dementia, Alzheimer's disease, diabetes, kidney disease, liver disease, etc.

In some embodiments, the methods of the present disclosure comprise treating the three dimensional pool with a small molecule compound from a library of small molecule compounds. For example, the small molecule compound may be from a library including but not limited to, MedChemExpress (a collection of 1280 structurally diverse, bioactive, and cell permeable compounds approved by the FDA and/or EMA; or a collection of 1600 structurally diverse, medicinally active, and cell permeable compounds that are or have been at some clinical stage), ChemDiv's master PPI library (20,000 diverse, computationally selected molecules comprising 7 subsets including natural product based, 3D mimetics, macrocycles, helix-turn mimetics, tripeptidomimetics, 3D diversity natural-product like, and Beyond flatland), the MayBridge collection (a set of 13,000 chemically diverse compounds), ChemBridge DIVERSet-CL (a collection of 50,000 small molecules with enhanced potential for therapeutic development), TargetMol (a collection of 3200 structurally diverse, medicinally active, and cell permeable compounds selected to expand and compliment NU-HTA's existing FDA approved and clinical collections), and/or any other small molecule compound library of interest.

The manner in which the three dimensional pool is treated with the small molecule compound will vary depending upon the context of the three dimensional pool. In the context of a three dimensional pool which is a xenograft comprising cells of different cell types, treating the three dimensional pool may comprise administering the small molecule compound to the recipient animal (e.g., mouse, rat, pig, or the like). The small molecule compound may be administered via a route of administration selected from oral (e.g., in tablet form, capsule form, liquid form, or the like), parenteral (e.g., by intravenous, intra-arterial, subcutaneous, intramuscular, or epidural injection), topical, intra-nasal, or intra-xenograft administration. In the context of a three dimensional pool which is an organoid, spheroid, or other 3D multicellular structure maintained and/or grown ex vivo or in vitro, treating the three dimensional pool may comprise addition of the small molecule compound to a cell culture medium in which the three dimensional pool is present. Suitable conditions for growing and/or maintaining the three dimensional pool prior to, during, and/or subsequent to treating the pool with the small molecule compound may vary. Such conditions may include growing and/or maintaining the three dimensional pool in a suitable container (e.g., a cell culture plate or well thereof), in suitable medium (e.g., cell culture medium, such as DMEM, RPMI, MEM, IMDM, DMEM/F-12, or the like) at a suitable temperature (e.g., 32° C.-42° C., such as 37° C.) and pH (e.g., pH 7.0-7.7, such as pH 7.4) in an environment having a suitable percentage of CO₂, e.g., 3% to 10%, such as 5%.

Subsequent to treatment of the three dimensional pool with the small molecule compound, the methods comprise dissociating cells of the treated three dimensional pool into single cells. A variety of suitable approaches for dissociating cells of the treated three dimensional pool into single cells may be employed. For example, when the three dimensional pool is an organoid, spheroid, or other 3D multicellular structure maintained and/or grown ex vivo or in vitro, the cells may be dissociated into single cells by digesting the three dimensional pool using Liberase™ enzyme blend (Millipore Sigma) in DMEM/F12 base media, and digested for 1 hour with rotation at 37° C. When the three dimensional pool is a xenograft, the xenograft (e.g., tumor xenograft) may be dissected from the sacrificed animal (e.g., from the flank of a mouse), chopped finely using a scalpel, resuspended in 1× Liberase™ enzyme blend in DMEM/F12 base media 10 U/uL DNAse I 1 mg/mL Collagenase IV, and digested for 1 hour with rotation at 37° C.

The methods of the present disclosure further comprise performing single cell ribonucleic acid (RNA) sequencing (sometimes referred to as “single-cell RNA-seq” or “scRNA-seq”) on the dissociated single cells and dissociated single cells from a control three dimensional pool not treated with the small molecule compound. RNA sequencing (RNA-seq) is a genomic approach for the detection and quantitative analysis of messenger RNA molecules in a biological sample and is useful for studying cellular responses. As a proxy for studying the proteome, some research has turned to protein-encoding, mRNA molecules (collectively termed the “transcriptome”), whose expression correlates well with cellular traits and changes in cellular state. scRNA-seq permits comparison of the transcriptomes of individual cells. A variety of suitable approaches for scRNA-seq are available, non-limiting examples of which include C1 (SMARTer) (e.g., see Pollen et al. (2014) Nat Biotechnol. 32:1053-8), Smart-seq2 (e.g., see Picelli et al. (2013) Nat Methods 10:1096-8), MATQ-seq (e.g., see Sheng et al. (2017) Nat Methods 14:267-70), MARS-seq (e.g., see Jaitin et al. (2014) Science 343:776-9), CEL-seq (e.g., see Hashimshony et al. (2012) Cell Rep. 2:666-73), Drop-seq (e.g., see Macosko et al. (2015) Cell 161:1202-14), InDrop (e.g., see Klein et al. (2015) Cell 161:1187-201), Chromium (e.g., see Zheng et al. (2017) Nat Commun. 8:14049), SEQ-well (e.g., see Gierahn et al. (2017) Nat Methods 14:395-8), SPLIT-seq (e.g., see Rosenberg et al. (2017) BioRxiv doi.org/10.1101/105163), and others. Further details regarding single-cell RNA-sequencing can be found, e.g., in Haque et al. (2017) Genome Med 9, 75.

In certain embodiments, performing scRNA-seq on the dissociated single cells comprising labeling the cells according to the Biolegend TotalSeq™-A protocol (www.biolegend.com/en-us/protocols/totalseq-a-antibodies-and-cell-hashing-with-10x-single-cell-3-reagent-kit-v3-3-1-protocol), performing the 10×3′ Chromium Single-Cell RNA-Sequencing Protocol (support. 10xgenomics.com/single-cell-gene-expression/library-prep/doc/user-guide-chromium-single-cell-3-reagent-kits-user-guide-v31-chemistry), and sequencing at about 300-400 M reads per 10× library and about 25 M reads per Biolegend TotalSeq™ Library. Data from the single cell RNA sequencing may be deconvoluted into single cell transcriptomes categorized by treatment (treated versus untreated with the small molecule compound) and cell type, e.g., using barcode sequence information.

Based on the categorized single cell transcriptomes, the methods further comprise assessing one or more therapeutic properties of the small molecule compound. The methods of the present disclosure find use in assessing a large variety of therapeutic properties of a small molecule compound. Non-limiting examples of such therapeutic properties include candidacy of the small molecule compound for combination therapy with a drug (combination therapy), mechanism of action (MoA) of the small molecule compound, candidacy of the small molecule compound for treatment of a disease subtype (e.g., for precision oncology including novel treatments for cancer/tumor subtypes), toxicity of the small molecule compound, mechanism of resistance/tolerance, drug repurposing for new indications not previously tested in the clinic, and many more.

According to some embodiments, the one or more therapeutic properties comprise candidacy of the small molecule compound for combination therapy with a drug, where such methods comprise, based on the single cell transcriptomes categorized by treatment and cell type, determining drug sensitivity for each cell line by counting the number of cells remaining in each condition, and calculating drug-induced gene expression changes for each cell line. Such methods further comprise assigning a weighted score for each gene based on its predicted relevance to drug sensitivity based on the calculated drug-induced gene expression changes for each cell line. Such methods further comprise predicting combination therapy targets based on the genes having weighted scores above a false discovery rate, where genes anti-correlated to drug sensitivity predict drug resistance and therefore represent candidate targets for combinatorial targeting.

Combination therapy discovery may comprise determining which cell types are sensitive to the compound and within those cell types determine the change in gene expression before and after treatment. Single cell-RNA sequencing may be performed with cell hashing and followed by demultiplexing each individual cell by using its single-nucleotide polymorphisms to assign cell line identity after assignment to a separate reference RNA sequencing dataset used to determine reference SNPs. Cell types sensitive to the compound may be determined by counting the number of cells remaining in each condition (drug, non-drug) for each cell line. Then, for each cell line, the difference in gene expression may be calculated before and after drug treatment, sometimes referred to herein as the “Single-Line Delta”. The aggregated Single-Line Deltas for the sensitive cell lines may be compared against the aggregated Single-Line Deltas for the insensitive cell lines to determine which genes are most up-regulated in the sensitive cells. These aggregated gene-expression changes may then be mapped onto online databases and using literature search determine which of these genes are druggable. Genes that are upregulated in response to the compound across all or most of the sensitive lines are identified as candidate combination therapy targets.

In certain embodiments, the one or more therapeutic properties comprise mechanism of action of the small molecule compound, where such methods comprise, based on the single cell transcriptomes categorized by treatment and cell type, determining drug sensitivity for each cell line by counting the number of cells remaining in each condition, determining drug-induced gene expression changes for each cell line, and aggregating the determined drug-induced gene expression changes across drug-sensitive cell lines. Such methods further comprise assigning a weighted score for each gene based on its predicted relevance to drug sensitivity based on the aggregated calculated drug-induced gene expression changes, identifying genes correlated with aggregated drug sensitivity as those having weighted scores above a false discovery rate, and predicting mechanism of action of the compound based on the genes correlated with the aggregated drug sensitivity.

Mechanism of action discovery may comprise experimentally dosing pools of cells using a serial dilution of small molecule compound concentrations, determining which cell types are sensitive to the compound, and within those cell types, determining the change in gene expression before and after treatment, and modeling the gene expression changes in sensitive versus insensitive cell lines as a function of small molecule compound concentration. First, the pools of cells may be experimentally subjected to a serial dilution of small molecule compound concentration ranges. Then, after single cell-RNA sequencing with cell hashing, each individual cell may be demultiplexed using its single-nucleotide polymorphisms (SNPs) to assign cell line identity after assignment to a separate reference RNA sequencing dataset used to determine reference SNPs. Cell types sensitive to the compound may be determined by counting the number of cells remaining in each condition (drug, non-drug) for each cell line. Then, for each cell line, the difference in gene expression may be calculated before and after drug treatment, sometimes referred to herein as the “Single-Line Delta”. The aggregated Single-Line Deltas for the sensitive cell lines may be compared against the aggregated Single-Line Deltas for the insensitive cell lines to determine which genes are most up-regulated in the sensitive cells. The gene expression changes may then be modeled as a function of the compound concentration used to determine what genes change as a direct function of drug concentration. These concentration-dependent gene-expression changes may then be mapped onto reference geneset databases to identify pathways into which these genes fall. In this model, negatively correlated genes as a function of drug concentration indicate the mechanism of action of the drug.

According to some embodiments, the one or more therapeutic properties comprise candidacy of the small molecule compound for treatment of a disease subtype, where such methods comprise, based on the single cell transcriptomes categorized by treatment and cell type, determining drug sensitivity for each cell line by counting the number of cells remaining in each condition, where each cell line is categorized by its genetic mutations and/or transcriptome signature. Such methods further comprise aggregating the determined drug sensitivity across cell lines, assigning a score for each mutation and/or transcriptome signature that predicts relevance to aggregated drug sensitivity using a variable selection regression algorithm, and predicting efficacy of the compound in a disease subtype based on the disease subtype having a score above a false discovery rate. In certain embodiments, the variable selection regression algorithm is a weighted lasso regression algorithm.

In recent years, therapies have been developed for targeting particular genetic variants of proteins. These particular genetic variants, or genetic subtypes, often are used to determine which drug(s) a patient should receive. However, there is currently no simple and high-throughput way to assess whether a drug developed for a particular genetic subtype can be used efficaciously against another subtype. The method described herein can simultaneously determine the relative sensitivity of a molecule against a range of genetic subtypes in in vivo, PDX (patient derived xenograft), in vitro, and ex vivo organoid model systems in one experiment. The method comprises pooling cells from multiple genetic subtypes. These mixed genetic subtype pools are then drugged using the small molecule. Single cell-RNA sequencing may be performed with cell hashing and followed by demultiplexing each individual cell by using its single-nucleotide polymorphisms to assign cell line identity after assignment to a separate reference RNA sequencing dataset used to determine reference SNPs. Cell types sensitive to the compound may be determined by counting the number of cells remaining in each condition (drug, non-drug) for each cell line. The cell lines may then be aggregated by their genetic subtypes and assessed for whether there is a shared sensitivity or resistance of different groups of lines categorized by subtype. Regression models (e.g., lasso regression models) may be trained on the mutations in each cell type to determine which genetic mutations predict the sensitivity calculated from the single-cell RNA sequencing deconvolved data. The mutations which effectively predict the sensitivity coefficient derived from the data indicate a potential target, and based on the sign of the coefficient of the model variable in the regression (e.g., lasso regression) for that mutation, it is possible to determine whether the mutation is a resistant (positive) or sensitizing (negative) mutation. The inventors have successfully employed this assay and modeling to demonstrate that it can predict known genetic subtype stratification as well as discover novel genetic subtypes which are sensitive to a molecule developed against a different genetic subtype. Subtype stratification, which may be defined as the ability to rank order and quantitatively estimate which genetic subtypes induce sensitivity or resistance to a small molecule, is able to be achieved in one pooled experiment using this method.

In one aspect, the disclosure provides, a balanced cell count culture comprising two or more different cell types that has been cultured for a time period wherein each of the at least two different cell types has a growth rate and wherein each cell type of the two or more different cell types are combined at a ratio inverse to the growth rate of each of the cell type of the two or more different cell types prior to culturing.

In one aspect, the disclosure provides, a balanced cell count culture comprising at least two or more different cell types, wherein a sample of from 0.2% to 10% by volume of the balanced cell count culture comprises at least 500 cells of each of the different cell types, wherein the sample is taken from the balanced cell count culture after the balanced cell count culture is cultured for a time period between 72 hours and 45 days after two or more cell types are combined to create a cell pool and inoculated in a culture media to obtain the balanced cell count culture.

In one aspect, the disclosure provides a balanced cell count culture comprising at least two or more different cell types, wherein each of the cell types is represented with at least 1×10³ cells in the culture and wherein at least two of the cell types are derived from different cancer tissues.

In one aspect, the disclosure provides, a balanced cell count culture comprising at least two or more different cell types wherein each of the cell types is represented with at least 1×10³ cells in the culture and wherein at least two of the cell types include cancer mutations that are different from each other.

In one aspect, the disclosure provides a balanced cell count culture comprising at least two or more different cell types wherein each of the cell types is represented with at least 1×10³ cells in the culture and wherein at least two of the cell types include cancer mutations that are different from each other.

In some embodiments, each of the two different cell types is represented with at least 1×10³ viable cells in the balanced cell count culture. In some embodiments, no cell type of the at least two different cell types in the balanced cell count culture outnumbers other cell types by 2 orders of magnitude or more. In some embodiments, the total number of each cell type of the at least two or more different cell types is within 2 orders of magnitude of each other in the balanced cell count culture. In some embodiments, the balanced cell count culture comprises from 2 to 500 different cell types. In some embodiments, the balanced cell count culture comprises from 2-500, 5-400, 6-300, 8-200, 10-100, 10-50, 2-30, 2-25, or 10-30 different cell types. In some embodiments, determining the representation of each cell type in a balanced cell count culture comprising multiple cell types comprises UMAP analysis. In some embodiments, UMAP analysis provides representation of different cell types in a balanced cell count culture as one or more clusters. In some embodiments, the balanced cell count culture comprises 2 or more, at least 2 or more, at least 3 or more, at least 4 or more, at least 5 or more, at least 6 or more, at least 7 or more, at least 8 or more, at least 9 or more, at least 10 or more, at least 11 or more, at least 12 or more, at least 13 or more, at least 14 or more, at least 15 or more, at least 16 or more, at least 17 or more, at least 18 or more, at least 19 or more, at least 20 or more different cell types. In some embodiments, the balanced cell count culture comprises 2, 3, 4, 5, 6, 7, 8, 9, 10, 11, 12, 13, 14, 15, 16, 17, 18, 19, 20, 21, 22, 23, 24,25, 26, 27, 28, 29, 30, 35, 40, 45, 50, 55, 60, 65, 70, 75, 80, 85, 90, 100 different cell types. In some embodiments, the balanced cell count culture is cultured for a time period between 6 hours and 45 days, between 12 hours and 40 days, between 24 hours and 35 days, between 72 hours and 30 days, between 96 hours and 20 days, between 120 hours and 15 days. In some embodiments, the balanced cell count culture is cultured for a time period of 72 hours. In some embodiments, the balanced cell count culture is cultured for a time period of 14 days. In some embodiments, each cell type of the two or more different cell types are combined at step (b) at a ratio inverse to the growth rate of each of the cell types as determined by the growth rate determination assay and ii) scaled to the total number of days for growth. In some embodiments a balanced cell count culture is a growth balanced culture (e.g., GENEVA pool). The terms balanced cell count culture, growth balanced culture, GENEVA pool, and GENEVA culture are used interchangeably in the spec. In some embodiments, the growth rate determination assay is a Calcein-AM growth assay or Cell Titer Glo growth assay. In some embodiments the growth rate is determined by a combination of Calcein-AM growth assay and Cell Titer Glo growth assay. In some embodiments, the growth rate is determined by the formula (Target Cell Number/Euler's Constant\hat{ }(Growth Rate*Number of Days Growth))/Cell Counts. In some embodiments, the growth rate is determined by the fold-increase in cell number. In some embodiments the fold increase is represented by Nf/NO, wherein Nf is the cell number at the end of culture time period and NO is the cell number at the beginning of culture period. In some embodiment, the growth rate is determined by r=ln(Nf/N0)/t, wherein “r” represents the growth rate, “t” represents the time of the assay, and In represents the natural logarithm. In some embodiments, the cells included in the balanced cell culture have a growth rate between 0.01 to 0.8, between 0.05 to 0.8, between 0.07 to 0.7, between 0.9 to 0.5, or between 0.1 to 0.4. In some embodiments, the cells from each cell type are included in a ratio such that the representation from each cell type was inversely proportional to their cell growth rates. In some embodiments, the growth rate is measured when target cell number equaled 10 million, 20 million, 30 million, 40 million, 50 million, 60 million, 70 million, 80 million, 90 million, or 100 million. In some embodiments, the growth rate is measured when target cell number equaled 100 million.

In some embodiments, growth Rate was taken from measurements performed determined by cell growth assay, and number of days growth equaled one. In some embodiments, growth Rate was taken from measurements performed determined by cell growth assay, and number of days growth equaled two. In some embodiments, growth Rate was taken from measurements performed determined by cell growth assay, and number of days growth equaled three. In some embodiments, growth Rate was taken from measurements performed determined by cell growth assay, and number of days growth equaled four. In some embodiments, growth Rate was taken from measurements performed determined by cell growth assay, and number of days growth equaled five. In some embodiments, growth Rate was taken from measurements performed determined by cell growth assay, and number of days growth equaled six. In some embodiments, growth Rate was taken from measurements performed determined by cell growth assay, and number of days growth equaled seven. In some embodiments, growth Rate was taken from measurements performed determined by cell growth assay, and number of days growth equaled ten. In some embodiments, the different cell types comprise cells with cancer mutations, cancer cells from one or more subjects, primary cells from one or more subjects, cells from an organ system, cells from a disease model, cells from a variety of cell lines or any combination thereof. In some embodiments, the different cell types comprise cells from subject having a disease. In some embodiments, the different cell types comprise cells from one or more subjects having a disease. In some embodiments, the different cell types comprise cells from a disease model, e.g., a organoid, e.g., a xenograft, e.g., a patient derived xenograft. In some embodiments, the disease is a neoplastic disease, e.g., cancer. In some embodiments, the cancer is selected from one or more of the cancer of head, neck, lung, skin, breast, blood, lymph, bone, soft tissue, brain, eye, reproductive system, circulatory system, digestive system, endocrine system, nervous systems, and of urinary system. In some embodiments, the cell lines are cancer cell lines. In some embodiments, the cancer cell lines may include but are not limited to one or more of H358, NCI-H23, H2122, H2030, SW1573, SK-LU-1, H441, CALU-1, H1792, H1373, H23, H358, H1299, H1975, SKMEL2, MEWO, SKMEL28, HTT144, A375, MIAPACA2, or A54.

In some embodiments, the balanced cell count culture is implanted in a model system, e.g., an in-vitro model system, in-vivo model system, or an ex-vivo model system. In some embodiments, the model system is a 2D in-vitro system. In some embodiments, the model system is a 3D in-vitro model system. In some embodiments, the model system is an 3D scaffolding system. In some embodiments, the model system is an ex-vivo model system, e.g., an organoid. In some embodiments, the model system is an in-vivo model system, e.g., an animal, e.g., a mammal, e.g., a mouse. In some embodiments, balanced cell count culture is implanted in a single mouse. In some embodiments, the implantation of the balanced cell count cultures create a mosaic tumor in an in-vivo system. In some embodiments, the implantation of the balanced cell count cultures create a mosaic tumor in a mouse. In embodiments, the disclosure provides a model system comprising a balanced cell count culture wherein the balanced cell count culture comprises multiple cell types. In some embodiments, the disclosure provides a model system comprising a mosaic tumor comprising multiple cell types. In some embodiments, the multiple cell types comprise cells of different physiological origin, cells from different subjects, cells from different organisms, cells from different tissues of the same organism, cells from the same tissue but from different organism, cell from different tissues or organs that are from different subjects. In some embodiments, the cells of different type comprise at least one different single nucleotide polymorphism from each other. In some embodiments, cells of different type comprises cancer mutations. In some embodiments, the cell can comprise identical cancer mutations. In some embodiments, the cells can comprise different cancer mutations. In some embodiments, the mutations comprise one or more of KRAS.G12C, EML4-ALK, TH21, TP53, PIK3CA, PTEN, APC, VHL, KRAS, MLL3, MLL2, ARID1A, PBRM1, NAV3, EGFR, NF1, PIK3R1, CDKN2A, GATA3, RB1, NOTCH1, FBXW7, CTNNB1, DNMT3A, MAP3K1, FLT3, MALAT1, TSHZ3, K EAP1, CDH1, ARHGAP35, CTCF, NFE2L2, SETBP1, BAP1, NPM1, RUNX1, NRAS, IDH1, TBX3, MA P2K4, RPL22, STK11, CRIPAK, CEBPA, KDM6A, EPHA3, AKT1, STAG2, BRAF, AR, AJUBA, EPPK1, TSHZ2, PIK3CG, SOX9, ATM, CDKN1B, WT1, HGF, KDM5C, PRX, ERBB4, MTOR, TLR4, U2AF1, AR ID5B, TET2, ATRX, MLL4, ELF3, BRCA1, LRRK2, POLQ, FOXA1, IDH2, CHEK2, KIT, HIST1H1C, SE TD2, PDGFRA, EP300, FGFR2, CCND1, EPHB6, SMAD4, FOXA2, USP9X, BRCA2, NFE2L3, FGFR 3, ASXL1, TGFBR2, SOX17, CDKN1A, B4GALT3, SF3B1, TAF1, PPP2R1A, CBFB, ATR, SIN3A, VEZ F1, HIST1H2BD, EIF4A2, CDK12, PHF6, SMC1A, PTPN11, ACVR1B, MAPK8IP1, H3F3C, NSD1, TB L1XR1, EGR3, ACVR2A, MECOM, LIFR, SMC3, NCOR1, RPL5, SMAD2, SPOP, AXIN2, MIR142, RA D21, ERCC2, CDKN2C, EZH2, PCBP1 mutations.

In one aspect, the present disclosure provides a method of preparing a balanced cell count culture with at least two or more different cell types, the method comprising:
(a) determining the growth rates for each cell type of the two or more different cell types;
(b) combining the two or more different cell types to create a cell pool, wherein the initial cell count of each of the cell types of the two or more different cell types added to the cell pool is determined based upon the growth rates of step (a); and
(c) culturing the cell pool of step (b) over a time period to create the balanced cell count culture, wherein a sample of from 0.2% to 10% by volume of the balanced cell count culture comprises at least 500 cells of each cell type of the two or more different cell types.

In some embodiments, the sample of step (c) comprises between 5,000-200,000 cells. In some embodiments the sample of step (c) comprises less than 200,000, less than 175,000, less than 150,000, than 140,000, less than 130,000, less than 120,000, less than 110,000, or less than 100,000 cells. In some embodiments, the sample of step (c) comprises no less than 500 viable cells of each cell type of the two or more different cell types. the different cell types comprise cells with cancer mutations, cancer cells from one or more subjects, primary cells from one or more subjects, cells from an organ system, cells from a disease model, cells from a variety of cell lines or any combination thereof. In some embodiments, the different cell types comprise cells from subject having a disease. In some embodiments, the different cell types comprise cells from one or more subjects having a disease. In some embodiments, the different cell types comprise cells from a disease model, e.g., a organoid, e.g., a xenograft, e.g., a patient derived xenograft. In some embodiments, the disease is a neoplastic disease, e.g., cancer. In some embodiments, the cancer is selected from one or more of the cancer of head, neck, lung, skin, breast, blood, lymph, bone, soft tissue, brain, eye, reproductive system, circulatory system, digestive system, endocrine system, nervous systems, and of urinary system In some embodiments, the sample of step (c) is taken at the end of the time period. In some embodiments, at least two or more samples of step (c) are taken at different time points during the time period. In some embodiments, the disclosure provides a method of correlating cells from the sample of step (c) of any one of claims 33-55 with the two or more cells of the cell pool of step (b) from the sample of step (c), performing steps further comprising:
(i) performing single cell RNA sequencing on one or more cells from the sample to identify single nucleotide polymorphisms in the one or more cells from the sample, and
(ii) comparing the single nucleotide polymorphisms of step (i) with single nucleotide polymorphisms of the two or more cells of the cell pool in step (b) thereby correlating cells from the sample of step (c) with the two or more cells of the cell pool of step (b).

In some embodiments, each of the two different cell types is represented with at least 1×10³ viable cells in the balanced cell count culture. In some embodiments, no cell type of the at least two different cell types in the balanced cell count culture outnumbers other cell types by 2 orders of magnitude or more. In some embodiments, the total number of each cell type of the at least two or more different cell types is within 2 orders of magnitude of each other in the balanced cell count culture.

In some embodiments, the balanced cell count culture comprises from 2 to 500 different cell types. In some embodiments, the balanced cell count culture comprises from 2-500, 5-400, 6-300, 8-200, 10-100, 10-50, 2-30, 2-25, or 10-30 different cell types. In some embodiments, the balanced cell count culture comprises 2 or more, at least 2 or more, at least 3 or more, at least 4 or more, at least 5 or more, at least 6 or more, at least 7 or more, at least 8 or more, at least 9 or more, at least 10 or more, at least 11 or more, at least 12 or more, at least 13 or more, at least 14 or more, at least 15 or more, at least 16 or more, at least 17 or more, at least 18 or more, at least 19 or more, at least 20 or more different cell types. In some embodiments, the balanced cell count culture comprises 2, 3, 4, 5, 6, 7, 8, 9, 10, 11, 12, 13, 14, 15, 16, 17, 18, 19, 20, 21, 22, 23, 24,25, 26, 27, 28, 29, 30, 35, 40, 45, 50, 55, 60, 65, 70, 75, 80, 85, 90, 100 different cell types.

In some embodiments, the balanced cell count culture is cultured for a time period between 6 hours and 45 days, between 12 hours and 40 days, between 24 hours and 35 days, between 72 hours and 30 days, between 96 hours and 20 days, between 120 hours and 15 days. In some embodiments, the balanced cell count culture is cultured for a time period of 72 hours. In some embodiments, the balanced cell count culture is cultured for a time period of 14 days. In some embodiments, balanced cell count culture comprises two or more different cell types, wherein each of the two or more different cell types is represented with at least 1×10³ cells in the culture and wherein at least two of the cell types include cancer mutations that are different from each other. In some embodiments, the disclosure provides a method of creating a mosaic tumor comprising at least two or more different cell types in an in-vivo model system. In some embodiments, the mosaic tumor is created by implanting a balanced cell count culture comprising two or more cells derived from cancer cell lines, from cancer tissues, and/or from subjects having cancer and implanting the balanced cell count culture in an in-vivo model system. In some embodiments the in-vivo model system is an animal, e.g., a mammal, e.g., a mouse.

In some embodiments, the balanced cell count culture is implanted in a model system, e.g., an in-vitro model system, in-vivo model system, or an ex-vivo model system. In one aspect the disclosure provides, a method of evaluating the impact of a candidate agent against two or more cell types, the method comprises preparing a balanced cell count culture; implanting the balanced cell count culture in a model system; treating the model system with a candidate agent over a duration of time; and evaluating the balanced cell count culture at the end of the duration of the time to determine phenotypic, genetic, and transcriptomic impact of the candidate agent on individual cells of the balanced cell count culture. In some embodiments, the present disclosure provides a method of evaluating the therapeutic efficacy of a candidate agent against individual cells of a mosaic tumor. In some embodiments, the therapeutic efficacy of a candidate agent is measured by treating the mosaic tumor by the candidate agent for a duration of time, evaluating the individual cells to determine phenotypic, genetic and transcriptomic expression of the individual cells of the mosaic tumor at the end of the duration of the time and determining the therapeutic efficacy of the candidate agent by comparing the phenotypic, genomic and transcriptomic expression of the individual cells of the mosaic tumor with phenotypic, genomic and transcriptomic expression of individual cells of an identical mosaic tumor that is not treated with the candidate agent.

In one aspect the disclosure provides, a method of evaluating the impact of a candidate agent against two or more cell types, the method comprising preparing a balanced cell count culture; implanting the balanced cell count culture in a model system; treating the model system with a candidate agent over a duration of time; and evaluating the balanced cell count culture at the end of the duration of the time to determine phenotypic, genetic, and transcriptomic impact of the candidate agent on individual cells of the balanced cell count culture. In some embodiments, the disclosure provides, a method of evaluating the impact of a candidate agent simultaneously against multiple cell types in an in-vivo system. In some embodiments, the disclosure provides, a method of evaluating the impact of a candidate agent simultaneously against multiple cell types in an in-vitro system. In some embodiments, the disclosure provides, a method of evaluating the impact of a candidate agent simultaneously against multiple cell types in an ex-vivo system. In some embodiments, the method comprises, preparing a balanced cell count culture; implanting the balanced cell count culture in a model system; treating the model system with a candidate agent over a duration of time evaluating the individual cells to determine phenotypic, genetic and transcriptomic expression of the individual cells of each of the multiple cell types at the end of the duration of the time, and determining impact of the candidate agent by comparing the phenotypic, genomic and transcriptomic expression of the individual cells of each of the multiple cell types in the model system with the phenotypic, genomic and transcriptomic expression of individual cells of each of multiple cell types of an identical model system that is not treated with the candidate agent.

In some embodiments, the disclosure provides a method of identifying a candidate agent target in a biological pathway, the method comprises, preparing a balanced cell count culture; implanting the balanced cell count culture in a model system; treating the model system with a candidate agent over a duration of time evaluating the individual cells to determine phenotypic, genetic and transcriptomic expression of the individual cells of each of the multiple cell types at the end of the duration of the time, and identifying the candidate agent target by comparing the phenotypic, genomic and transcriptomic expression of the individual cells of each of the multiple cell types in the model system with the phenotypic, genomic and transcriptomic expression of individual cells of each of multiple cell types of an identical model system that is not treated with the candidate agent. In some embodiments, the disclosure provides a method of identifying a subject sub-population sensitive to a candidate agent. The method comprises, preparing a balanced cell count culture; implanting the balanced cell count culture in a model system; treating the model system with a candidate agent over a duration of time evaluating the individual cells to determine phenotypic, genetic and transcriptomic expression of the individual cells of each of the multiple cell types at the end of the duration of the time, and identifying the subject sub-population sensitive to the candidate agent based on the evaluation of the phenotypic, genetic and transcriptomic impact of the candidate agent on individual cells of the balanced cell count culture. In some embodiments, the disclosure provides a method of identifying the time point when a subject subpopulation become resistant to a drug by determining phenotypic, genetic and transcriptomic expression of an individual cell from the subjects using a method described herein. In some embodiments, the disclosure provides a method of determining a time point when a subject subpopulation becomes to a therapeutic treatment by a candidate agent by determining phenotypic, genetic and transcriptomic expression of an individual cell from the subjects using a method described herein. In some embodiments, the disclosure provides a method of determining a personalized treatment regime from a subject population by determining the effect of one or more therapeutic agents on the phenotypic, genetic and transcriptomic expression of an individual cell from the subjects using a method described herein and determining a treatment regimen based on the phenotypic, genetic and transcriptomic expression of an individual cell from the subjects.

In some embodiments, the disclosure provides a method of identifying the efficacy of a combination therapy by preparing a balanced cell count culture; implanting the balanced cell count culture in a model system; treating the model system with two or more candidate agents in combination over a duration of time evaluating the individual cells to determine phenotypic, genetic and transcriptomic expression of the individual cells of each of the multiple cell types at the end of the duration of the time, and identifying the efficacy of the combination treatment by the effect of the combination treatment on the individual cells. In some embodiments, the method comprises treating with a first candidate agent and treating with a second candidate agent. In some embodiments, the treatment with the first candidate agent and the second candidate agent is continuous. In some embodiments, the treatment with the first candidate agent and the second candidate agent is consecutive. In some embodiments, the method optionally comprises treating with a third candidate agent.

In some embodiments, the model system, is an in-vitro model system, an in-vivo model system, or an ex-vivo model system. In some embodiments, the model system is a 2D in-vitro system. In some embodiments, the model system is a 3D in-vitro model system. In some embodiments, the model system is an 3D scaffolding system. In some embodiments, the model system is an ex-vivo model system, e.g., an organoid. In some embodiments, the model system is an in-vivo model system, e.g., a animal, e.g., a mammal, e.g., a mouse.

In some embodiments, the duration of time is about 1 hour, about 2 hours, about 4 hours, about 6 hours, about 10 hours, about 16 hours, about 24 hours, about 36 hours, about 48 hours, about 60 hours, about 72 hours, about 84 hours, about 96 hours, about 120 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 20 days, about 24 days, about 25 days, about 30 days, about 35 days, about 40 days, or about 45 days. In some embodiments, the treatment is intermittent. In some embodiments treatment is continuous.

In some embodiments, the candidate agent is an agent that can cause a therapeutic perturbation. In some embodiments, the candidate agent is selected from a small molecule, an antibody, a peptide, a gene editor, or a nucleic acid aptamer. In some embodiments, the small molecule is a KRAS.G12C inhibitor, e.g., ARS-1620, AMG510, or MRTX849. In some embodiments, the candidate agent is an inhibitor of a biological pathway. In some embodiments, the candidate agent is an activator of a biological pathway. In embodiments the candidate agent is selected from one or more of ARS-1620, AMG510, Galunisertib, MRTX849, INK128, and Antimycin.

In some embodiments, evaluating phenotypic changes comprises counting the number of viable individual cells of each of the cell types of the two or more different cell types at the end of the duration of the time. In some embodiments, evaluating transcriptomic impact comprises determining single-cell transcriptome profiles of cells of in the balanced cell count culture at the end of the duration of the time. In some embodiments, evaluating genetic impact comprises single cell RNA sequencing of cells in the balanced cell count culture at the end of the duration of the time. In some embodiments, the effect of the candidate agent on individual cells of the balanced cell count culture is assessed by calculating gene expression for individual cells of the balanced cell count culture treated by the candidate agent and compare the gene expression with the gene expression for individual cells of an identical balanced cell count culture that is not treated by the candidate agent. In some embodiments, the effect of the candidate agent on individual cells of the balanced cell count culture is assessed by determining transcriptomic expression for individual cells of the balanced cell count culture treated by the candidate agent and compare the transcriptomic expression with the gene expression for individual cells of an identical balanced cell count culture that is not treated by the candidate agent. In some embodiments, the effect of the candidate agent on individual cells of the balanced cell count culture is assessed by counting the number of viable individual cells of each of the cell types of the two or more different cell types in the balanced cell count culture in the treated by the candidate agent and comparing the number of viable individual cells of each of the cell types of the two or more different cell types in an identical balanced cell count culture that is not treated by the candidate agent. In some embodiments, the assessment includes determination of one or more of genetic impact, phenotypic impact, and transcriptomic impact.

### Computer Readable Media and Systems

Aspects of the present disclosure also include computer readable media and systems. The computer readable media and systems find use in a variety of contexts, including but not limited to, in practicing the methods of the present disclosure.

In certain aspects, provided are one or more non-transitory computer-readable media comprising instructions stored thereon. When executed by one or more processors, the instructions cause the one or more processors to deconvolute single cell RNA sequencing data into single cell transcriptomes categorized by treatment and cell type. The single cell RNA sequencing data was produced by performing single cell RNA sequencing on dissociated single cells from a three dimensional pool (e.g., a xenograft, an organoid, or the like) of different cell types treated with a small molecule compound, and also on dissociated single cells from a control three dimensional pool of different cell types not treated with the small molecule compound. When executed by the one or more processors, the instructions further cause the one or more processors to assess one or more therapeutic properties of the small molecule compound based on the categorized single cell transcriptomes.

In certain embodiments, the instructions cause the one or more processors to assess one or more therapeutic properties of the small molecule compound based on the categorized single cell transcriptomes, where the one or more therapeutic properties comprise candidacy of the small molecule compound for combination therapy with a drug. According to some embodiments, the instructions cause the one or more processors to, based on the single cell transcriptomes categorized by treatment and cell type, calculate drug-induced gene expression changes for each cell line, assign a weighted score for each gene based on its predicted relevance to drug sensitivity based on the calculated drug-induced gene expression changes for each cell line, and predict combination therapy targets based on the genes having weighted scores above a false discovery rate, where genes anti-correlated to drug sensitivity predict drug resistance and therefore represent candidate targets for combinatorial targeting.

According to some embodiments, the instructions cause the one or more processors to assess one or more therapeutic properties of the small molecule compound based on the categorized single cell transcriptomes, where the one or more therapeutic properties comprise mechanism of action of the small molecule compound. In certain embodiments, the instructions cause the one or more processors to, based on the single cell transcriptomes categorized by treatment and cell type, determine drug-induced gene expression changes for each cell line, aggregate the determined drug-induced gene expression changes across drug-sensitive cell lines, assign a weighted score for each gene based on its predicted relevance to drug sensitivity based on the aggregated calculated drug-induced gene expression changes, identify genes correlated with aggregated drug sensitivity as those having weighted scores above a false discovery rate, and predict mechanism of action of the compound based on the genes correlated with the aggregated drug sensitivity.

In certain embodiments, the instructions cause the one or more processors to assess one or more therapeutic properties of the small molecule compound based on the categorized single cell transcriptomes, where the one or more therapeutic properties comprise candidacy of the small molecule compound for treatment of a disease subtype. According to some embodiments, the instructions cause the one or more processors to, based on the single cell transcriptomes categorized by treatment and cell type, aggregate drug sensitivity across cell lines, wherein drug sensitivity is determined for each cell line by counting the number of cells remaining in each condition, wherein each cell line is categorized by its genetic mutations and/or transcriptome signature. The instructions cause the one or more processors to assign a score for each mutation and/or transcriptome signature that predicts relevance to aggregated drug sensitivity using a variable selection regression algorithm, and predict efficacy of the compound in a disease subtype based on the disease subtype having a score above a false discovery rate. In certain embodiments, the variable selection regression algorithm is a weighted lasso regression algorithm.

In certain aspects, provided are systems for assessing one or more therapeutic properties of a small molecule compound. Such systems comprise one or more processors and one or more non-transitory computer-readable media comprising instructions stored thereon. When executed by one or more processors, the instructions cause the one or more processors to deconvolute single cell RNA sequencing data into single cell transcriptomes categorized by treatment and cell type. The single cell RNA sequencing data was produced by performing single cell RNA sequencing on dissociated single cells from a three dimensional pool (e.g., a xenograft, an organoid, or the like) of different cell types treated with a small molecule compound, and also on dissociated single cells from a control three dimensional pool of different cell types not treated with the small molecule compound. When executed by the one or more processors, the instructions further cause the one or more processors to assess one or more therapeutic properties of the small molecule compound based on the categorized single cell transcriptomes.

In certain embodiments, the instructions of the one or more computer readable media of the systems of the present disclosure cause the one or more processors to assess one or more therapeutic properties of the small molecule compound based on the categorized single cell transcriptomes, where the one or more therapeutic properties comprise candidacy of the small molecule compound for combination therapy with a drug, mechanism of action of the small molecule compound, candidacy of the small molecule compound for treatment of a disease subtype, or any combination thereof. Examples of instructions of such non-transitory computer-readable media for performing these and other types of assessments are described hereinabove and not reiterated herein for purposes of brevity.

A variety of processor-based systems may be employed to implement the embodiments of the present disclosure. Such systems may include system architecture wherein the components of the system are in electrical communication with each other using a bus. System architecture can include a processing unit (CPU or processor), as well as a cache, that are variously coupled to the system bus. The bus couples various system components including system memory (e.g., read only memory (ROM) and random access memory (RAM), to the processor.

System architecture can include a cache of high-speed memory connected directly with, in close proximity to, or integrated as part of the processor. System architecture can copy data from the memory and/or the storage device to the cache for quick access by the processor. In this way, the cache can provide a performance boost that avoids processor delays while waiting for data. These and other modules can control or be configured to control the processor to perform various actions. Other system memory may be available for use as well. Memory can include multiple different types of memory with different performance characteristics. Processor can include any general purpose processor and a hardware module or software module, such as first, second and third modules stored in the storage device, configured to control the processor as well as a special-purpose processor where software instructions are incorporated into the actual processor design. The processor may essentially be a completely self-contained computing system, containing multiple cores or processors, a bus, memory controller, cache, etc. A multi-core processor may be symmetric or asymmetric.

To enable user interaction with the computing system architecture, an input device can represent any number of input mechanisms, such as a microphone for speech, a touch-sensitive screen for gesture or graphical input, keyboard, mouse, motion input, speech and so forth. An output device can also be one or more of a number of output mechanisms. In some instances, multimodal systems can enable a user to provide multiple types of input to communicate with the computing system architecture. A communications interface can generally govern and manage the user input and system output. There is no restriction on operating on any particular hardware arrangement and therefore the basic features here may easily be substituted for improved hardware or firmware arrangements as they are developed.

The storage device is typically a non-volatile memory and can be a hard disk or other types of computer-readable media which can store data that are accessible by a computer, such as magnetic cassettes, flash memory cards, solid state memory devices, digital versatile disks, cartridges, random access memories (RAMs), read only memory (ROM), and hybrids thereof.

The storage device can include software modules for controlling the processor. Other hardware or software modules are contemplated. The storage device can be connected to the system bus. In one aspect, a hardware module that performs a particular function can include the software component stored in a computer-readable medium in connection with the necessary hardware components, such as the processor, bus, output device, and so forth, to carry out various functions of the disclosed technology.

Embodiments within the scope of the present disclosure may also include tangible and/or non-transitory computer-readable storage media or devices for carrying or having computer-executable instructions or data structures stored thereon. Such tangible computer-readable storage devices can be any available device that can be accessed by a general purpose or special purpose computer, including the functional design of any special purpose processor as described above. By way of example, and not limitation, such tangible computer-readable devices can include RAM, ROM, EEPROM, CD-ROM or other optical disk storage, magnetic disk storage or other magnetic storage devices, or any other device which can be used to carry or store desired program code in the form of computer-executable instructions, data structures, or processor chip design. When information or instructions are provided via a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable storage devices.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform tasks or implement abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

Other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

The following examples are offered by way of illustration and not by way of limitation.

### EXPERIMENTAL

### Example 1—Creation of a 3D Heterogeneous Cell Pool

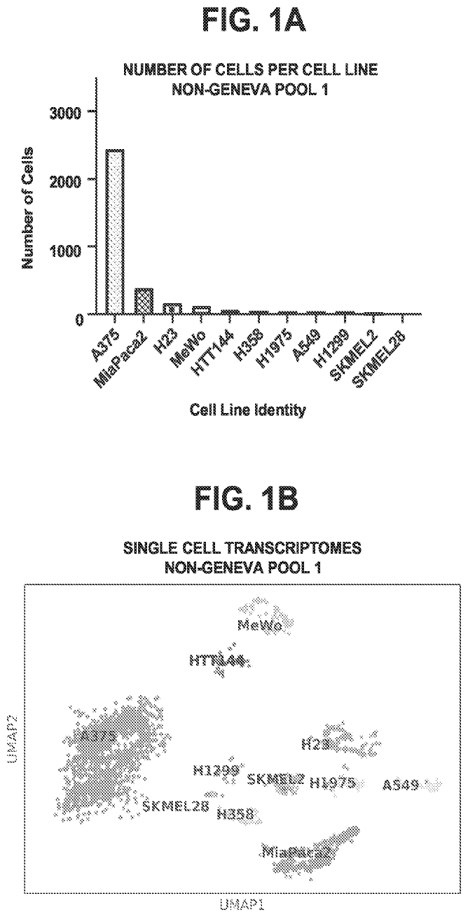

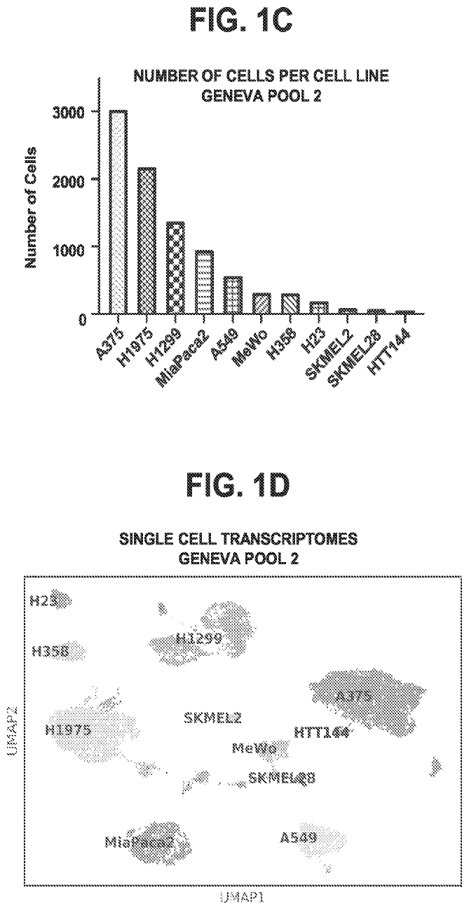

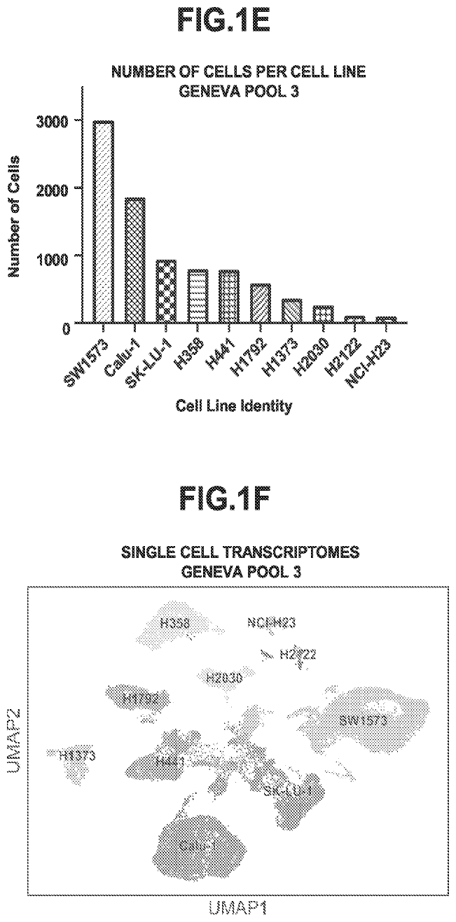

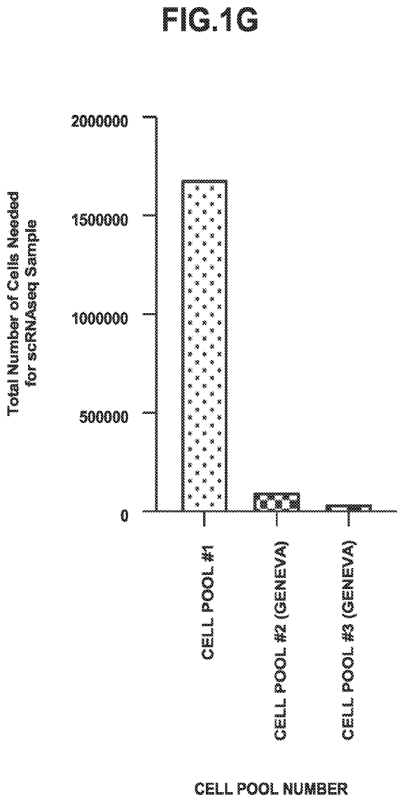

This example is directed to creation of a 3D heterogeneous cell pool. To create a pool of heterogeneous cell lines that would produce equivalent numbers of cells at harvest after a long duration of growth, a pool of eleven human cell lines from different people was utilized. A pool was created where the number of cells at time of pooling was equivalent for each line. Cells were then subjected to a course of seven days of growth in cell culture. After seven days, the pools were harvested, and single-cell RNA sequencing was performed on a 10× Chromium platform to obtain single-cell RNA sequencing Illumina fragment libraries. The libraries were sequenced on Illumina instruments and the reads were aligned to obtain single-cell gene expression profiles. Using demultiplexing software, single-cell data packages were then resolved into their patient of origin (demuxlet, freemuxlet). It was determined that over 90% of the single-cells were from the cell line A375 while the other ten cell lines made up<10% of the remaining cell counts. Accurate transcriptome states were unable to be obtained for several of the cell lines because of the few/limited number of cells, specifically H358, H1975, A549, H1299, SKMEL2, and SKMEL28 (FIG. 1A-B).

### Example 2—Creation of Growth Rate Balanced 3D Heterogeneous Cell Pools (GENEVA)

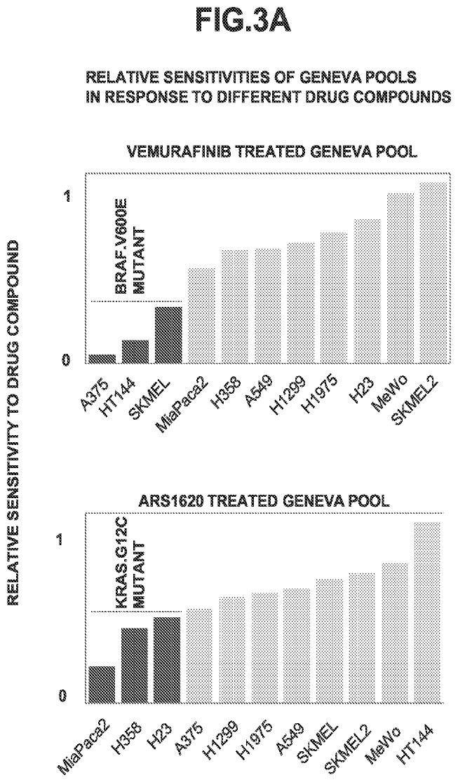

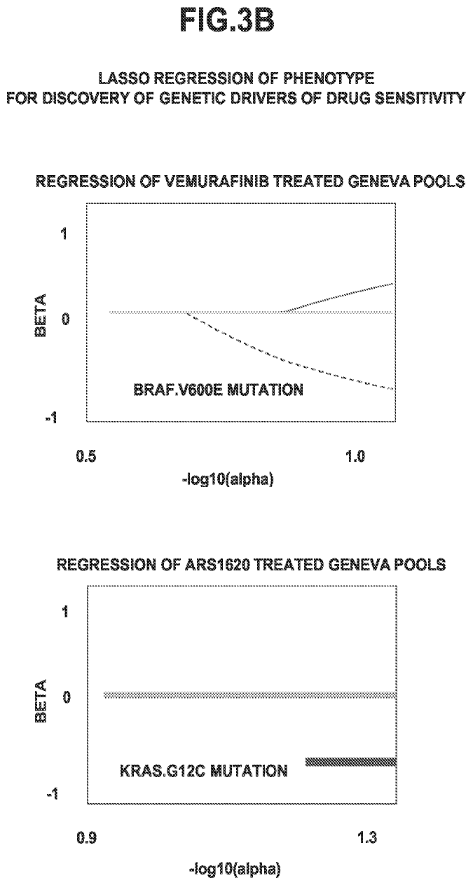

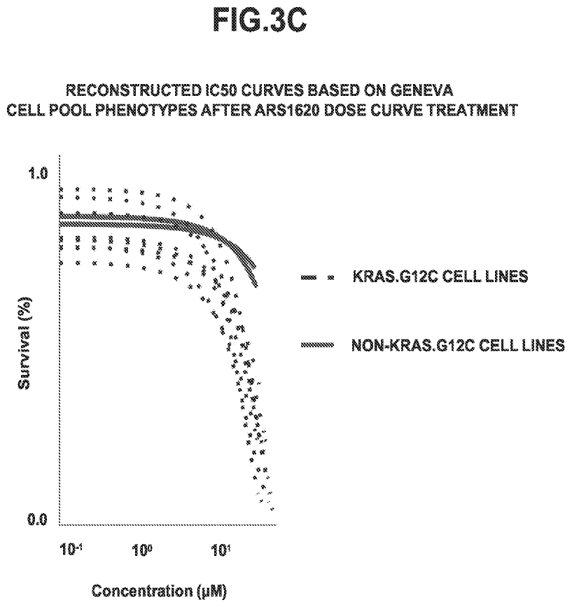

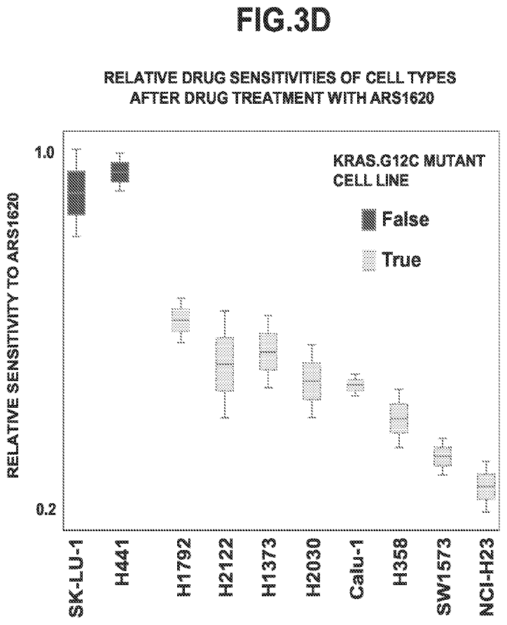

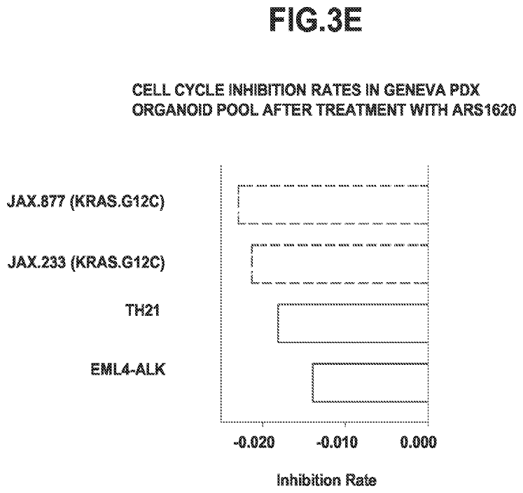

This example is directed to creation of a growth rate balanced 3D heterogeneous cell pool. The growth rates of the cell lines comprising the pool were measured individually using a cell growth rate assay prior to adding the cells for each cell line in a pool (H23, H358, H1299, H1975, SKMEL2, MEWO, SKMEL28, HTT144, A375, MIAPACA2, A549). Using these growth rates, cell numbers were then balanced at pooling at a ratio i) inverse to the growth rate of the cell lines as determined by growth assay and ii) scaled to the number of days for longitudinal growth. After pooling, growth, harvest, and single-cell RNA sequencing and analysis, the pool of cell lines balanced in this manner produced a more even distribution of cell types between different cell lines and allowed for accurate single-cell transcriptome profiles from all cell lines included in the pool compared to the pool obtained in Example 1 (FIG. 2A-B). To verify that the pools created by inversely balancing growth rates were able to produce more evenly balanced pools, an experiment was performed with different cell lines comprising the pool while retaining the methodology of inverse growth rate balancing based on time (H358, NCI-H23, H2122, H2030, SW1573, SK-LU-1, H441, CALU-1, H1792, H1373). It was found that this third pool was also able to produce more evenly distributed numbers of cells across different cell lines allowing for accurate transcriptional inference of expression profiles after a long course of pooled growth (FIG. 3A-B).

These experiments with inverse growth rate and time balancing demonstrated that it was essential to measure growth rates and follow a strict balancing approach to produce cell pools that would be able to grow and be evenly represented at a defined time of harvest. Sampling small samples from these pools (<100,000 cell samples) contained representatives from all cell lines of origin, while for pools that were not created by growth rate inverse balancing required million-cell samples to contain representatives from all cell lines of origin (FIG. 1G).

### Growth Rate Measurements for Determination of GENEVA Inclusion Criteria

Adherent cell lines were propagated in RPMI supplemented with 10% fetal bovine serum (FBS) for two passages after thawing. Cell lines were then dissociated using Trypsin (0.25%) into a single cell suspension. Cell counts were then obtained using an electronic cell counting instrument and 5,000 cells of each cell line was seeded individually into wells of two identical 96 well plates. Two hours after seeding, one plate was assayed for cellular viability using cell-titer-glow (CTG) reagent from Promega at 2 hours. Briefly, media in the 96-well assay plate was removed by decanting and 50 μL of CTG reagent was added directly to the plate containing cells. After 30 minutes of incubation at 37C, the plate was then read at 100V on a 96-well compatible luminometer to measure cellular viability. After 72 hours of growth in RPMI 10% FBS, media was removed by decanting, and 50 μL of CTG reagent was added directly to the plate containing cells. After 30 minutes of incubation at 37° C., the plate was then read at 100V on a 96-well compatible luminometer to measure cellular viability. To measure growth rate, the raw luminescence signal at 72 hours was divided by the luminescence signal at 2 hours. This ratio was estimated to be the fold-increase in cell number, hereafter referred to as Nf/NO. Growth rate was then calculated for each cell line using the formula: r=ln(Nf/N0)/t where “r” represents the growth rate, “t” represents the time of the assay, and In represents the natural logarithm. Growth rates for all cell lines comprising the pools were determined in this fashion and cells with only growth rates greater than 0.1 and less than 0.4 were determined to be viable candidates for inclusion in GENEVA pools. Cell lines with growth rates outside of these parameters were omitted from further consideration in GENEVA pools.

### GENEVA Cell Pool Creation According to Inverse Growth Rate and Time Ratios

Pools were then created by including cells from each cell type such that the representation from each cell type was inversely proportional to their cell growth rates. Cell counts were taken and the following calculation was performed to determine the volume of cell suspension to seed into the GENEVA pool: (Target Cell Number/Euler's Constant\hat{ }(Growth Rate*Number of Days Growth))/Cell Counts where the target cell number equaled one hundred million, Growth Rate was taken from measurements performed determined by cell growth assay, and number of days growth equaled seven. Using this formula, the number of cells to add into the GENEVA cell pool was calculated and cells were combined into a single suspension. Cells were then grown for seven days in cell culture in RPMI, 10% FBS. The cells were harvested by dissociation with 0.25% Trypsin into single-cell suspension. Following estimation of cellular viability and dilution of cells to 2000 cells/uL, single cells were then loaded with “GEM Generation Reagents” as specified in the “10× Chromium v3.0” protocol”. Further processing of single cell suspension was performed as described in the 10× Chromium method. Illumina Sequencing was performed to obtain 25,000 reads per cell.

### Generation of Single-Nucleotide Reference Panels for Deconvolution of Single-Cells to Patient of Origin

Following from above, cell lines that were determined to be viable for GENEVA pools were then individually seeded into 6-well cell culture plates for growth as follows: Cell lines were dissociated using Trypsin (0.25%) into a single cell suspension. Cell counts were then obtained using an electronic cell counting instrument and 200,000 cells of each cell line was seeded individually into wells of a 6-well cell culture plate. 2 mLs of media were then added to serve as growth media. Following two days of growth, 6-well plates were then harvested for RNA extraction by decanting the media and addition of 400 ul of Trizol RNA Extraction Reagent directly to the cells. Extraction of RNA was using the ThermoFisher Trizol RNA Extraction Method. RNA extracted using this procedure was then transferred to RNAse-free microcentrifuge tubes and assayed for purity by aliquoting 2 uLs of the RNA solution onto a Nanodrop instrument. Illumina compatible DNA libraries was prepared using “Quantseq” kit from Lexogen and sequenced on Illumina instruments.

Pooled Genetic Signature Data Generation from GENEVA Suitable Cell Lines

Cell lines that were determined to be viable for GENEVA pools were then prepared into an evenly distributed pool mixture of cell lines for GENEVA pooled genetic signature data generation: Cell lines were dissociated using Trypsin (0.25%) into a single cell suspension. Cell counts were then obtained using an electronic cell counting instrument and 500,000 cells of each cell line was seeded individually into one 50 ml conical tube containing 5 mLs of 1× Phosphate Buffered Saline (PBS) at 4 degrees Celsius (4C). After all cell lines were added into the tube of pooled GENEVA cell lines centrifugation at 400 g for 10 minutes at 4C was performed. Supernatant was decanted and the cell pellet was resuspended with 5 mL 1×PBS. Cells were spun again at 400 g for 10 minutes at 4C, and supernatant was decanted and the pellet was resuspended with 2.5 mL 1×PBS. Cells were spun again at 400 g for 10 minutes at 4C, and supernatant was decanted and the pellet was resuspended with 0.5 mL 1×PBS and resulting solution was filtered through a 45 micron filter tube to obtain a single-cell suspension of pooled cell lines free of contaminating Trypsin and fetal bovine serum. This solution was then counted using an automated cell counter and diluted to 2000 cells/μL. Live cell estimation was also performed by obtaining a count with 1:1 of Trypan Blue, 1×PBS: GENEVA pool, 1×PBS. Viability of the pool if greater than 85% was allowed to proceed for single-cell RNA sequencing preparation. Following estimation of cellular viability and dilution of cells to 2000 cells/μL, single cells were then loaded with “GEM Generation Reagents” as specified in the “10× Chromium v3.0” protocol and resulting Illumina libraries were sequenced to a depth of 25,000 reads per cell.

### Creation of GENEVA Relevant Single-Nucleotide Polymorphisms List by Integration of i) Individual Cell Line Genetic Signature Dataset and ii) Pooled GENEVA Genetic Signature Dataset

Sequencing data from single-nucleotide reference panels was first computationally deconstructed to obtain clean single-nucleotide polymorphism calls from RNA sequencing data. Sequencing files (fastq format) were trimmed on a per read basis to remove poly-adenylation and Truseq Illumina sequencing adaptor contamination, aligned using the “bwa” whole genome alignment tool, sorted and formatted using the “samtools” tool, and deduplicated by unique molecular identifiers using the “umi_tools” tool. Individual reads, now cleaned of sequence artifacts and aligned fully to a genomic location, were then stacked by location using the tool “samtools mpileup” and then converted to bcf and finally a single merged vcf data structure format using the “bcftools” and “vcf-merge” tool. Sequencing data from section 1.d (above) was computationally deconstructed to obtain full genomic alignment data from single-cell transcriptomes. Sequencing files (fastq format) were processed to aligned “.bam” format using the “cellranger” tool from 10× Genomics as. The resulting “.bam” file was then used for downstream data integration in conjunction with the individual cell line vcf files. A merged “.vcf” file comprising all detected SNP mutations from individual cell lines (section 1.d.i) and a “.bam” file containing all SNP mutations from a GENEVA pool created from those same lines produced using single-cell RNA sequencing methods were used as input for data integration for selection and filtering of relevant SNPs used for downstream GENEVA demultiplexing by SNPs. Data integration was performed with the intent of removing computationally non-informative SNPs that would prevent accurate genotyping of single-cells from a GENEVA pool back to their cell line of origin. The merged “.vcf” file was intersected with the “.bam” file with a filtering criteria of >250 reads per loci to allow for only high-confidence reads mapping between both datasets using the “bedtools intersect” tool. These SNPs were then filtered further using a recursive algorithm that integrated the tool “demuxlet” as a way of measuring vcf algorithm improvement. The algorithm removed each SNP individually from the merged vcf file to generate a data-subtracted “.vcf” file as test subject. This data-subtracted “.vcf” file was then used in conjunction with the “.bam” file to run demuxlet which provided the relative singlet ratio, a measure of demultiplexing by SNP fidelity. By iteratively testing the individual contribution of single SNPs on overall demultiplexing by SNP fidelity, a limited list of high-quality SNPs was arrived at and used as high-quality reference SNPs for downstream demultiplexing and further GENEVA experimentation using this specific GENEVA pool.

### Example 3—Comparison of Ability to Perform Long-Duration Pool Growth and Drug Treatment on Non-Growth Balanced and Growth Balanced 3D Heterogeneous Cell Pools

This example is directed at growing pools for greater than 72 hours while treating them with drug compounds to allow for understanding of long-term drug impact on cells.

Using a 14 day long-term treatment duration, pools were balanced as described in Example 2 by setting the “Number of Days Growth” variable equal to fourteen in the following equation:

(Target Cell Density/Euler's Constant\hat{ }(Growth Rate*Number of Days Growth))/Cell Counts

Heterogeneous cell pools transplanted in various model systems were created. Small samples of the treated pools (˜1% of total cells) contained enough cells from all cells of origin to accurately assess the impact of the drug on that cell line over fourteen days of treatment (FIG. 1C,D,E,F). In contrast 90% of the cells in a non-growth balanced heterogeneous 3D cell pool were from one cell type (FIG. 1A,B).

### Example 4—Creation of In-Vivo or Ex-Vivo 3D Model Systems Using GENEVA Cell Pools

This example is directed at assessing the impact of long-term treatment using drug therapeutics on complex model systems such as in-vivo mouse models and in-vitro 3D model systems.

Using growth-rate balanced heterogeneous cell pools adjusted for long time-course drug treatment, pools were created from four human patient-derived xenograft (PDX) models and implanted as a pooled tumor in a flank xenograft mouse model. The pooled tumors were drugged in mice by oral dosing by gavage for fourteen days with the molecule ARS-1620. After the treatment interval, tumors were harvested from mice, and single-cell RNA sequencing, genetic demultiplexing (as illustrated in Example 2), and sample hashing using barcoded antibodies was performed. Sufficient numbers of cells were observed from each PDX genetic background and drug treatment condition (FIG. 2C,D) for inference of drug phenotype and drug changes to the transcriptome. Pools from four PDX models were created for implantation for a fourteen day drug treatment in a 3D Organoid model system. Cell pools were treated with three increasing doses of ARS-1620 (0.4 uM, 1.6 uM, 25.0 uM) and one vehicle condition for fourteen days. After the treatment interval, tumors from 3D Organoids were harvested and single-cell RNA sequencing, genetic demultiplexing and sample hashing were performed. Sufficient numbers of cells were observed from each PDX genetic background and drug treatment condition (FIG. 2A,B) in the organoid models. Pools were further created using human cell lines for implantation in in-vivo flank xenograft mouse models using the method described above.

### Implantation, Drugging, and Harvest of 3D Organoid GENEVA Models

5 mL of Matrigel Basement Membrane reagent was added to the GENEVA pool prepared by growth rate balancing for a final concentration of 5M/mL. Fifty microliters of this solution was transferred into a 6-well plate and incubated at 37 C for 30 minutes. Organoid media (Advanced DMEM/F12 base, 1×N-2, 1×B-27, 10 mM HEPES, 2 mM L-Glutamine, 1× Pen-Strep, 500 ng/ml FGF10, 1% FBS) was added onto cells for overnight recovery. Sixteen hours later organoids were drugged with the drug compound diluted in organoid media. Organoids were grown for fourteen days in drug media with fresh drug compound media added every 72 hours. On the fourteenth day organoids were harvested by manual dissociation and resuspended in 10 mg/ml Liberase™ Cell Dissociation Reagent in 1:1 DMEM: F12 cell media, DNAse I (10 U/uL). Organoids were incubated in a 37C incubator with shaking at 600 RPMs for 45 minutes for enzymatic dissociation and then spun down at 800 g for 5 minutes at 4C. Dissociated organoids were resuspended in 100 μL 1×PBS.

### Implantation, Drugging, and Harvest of In Vivo GENEVA Models

2 mL of Matrigel Basement Membrane reagent was added to the GENEVA pool prepared by growth rate balancing for a final concentration of 20M/mL. One hundred microliters of this solution was injected into NSG mice in a flank xenograft injection. Twenty-four hours later, mice harboring GENEVA tumors were drugged with the drug compound in a vehicle solution of 5% DMSO, 95% Labrasol. Mice were dosed by oral gavage four fourteen days with five days on, two days off. On the fourteenth day, mice were sacrificed, tumors harvested by homogenization with surgical shears and resuspended in 5 mg/mL Liberase™ Cell Dissociation Reagent in 1:1 DMEM: F12 cell media, DNAse I (10 U/uL). Tumors were incubated in a 37° C. incubator with shaking at 600 RPMs for 45 minutes for enzymatic dissociation and then spun down at 800 g for 5 minutes at 4° C. Dissociated tumors were resuspended in 100 uL 1×PBS.

### Example 5—Identification of Cell Origin Through Single RNA Sequencing and Transcriptome Analysis

This example is directed at assignment of single cells to their patient or cell line of origin. Using data from GENEVA experiments, fastq sequencing files corresponding to a GENEVA pooled mRNA library and the individual generated reference VCF file with known genotypes were obtained. The GENEVA mRNA library was deconvolved using the tools freemuxlet and demuxlet (github.com/statgen/popscle). A consensus approach was taken to match clusters called as unique individuals by freemuxlet and known populations from the demuxlet approach. This genetics-alone approach was then integrated with transcriptome information. Cells were 35 clustered using the GENEVA mRNA library data and clusters with a leiden sparsity factor greater than or equal to ten were called. A maximum likelihood match was then assigned between each transcriptionally defined leiden cluster and each genetics-alone population to obtain the percent frequency representation of each transcriptome cluster. A >70% cutoff was implemented to obtain clusters that had high accuracy between transcriptome and genetics and removed clusters below this threshold.

### Deconvolution by Genetics

Single cells were deconvolved based on single-cell RNA based single-nucleotide polymorphism calls. Freemuxlet was run with cluster numbers fixed at the number of cell types used as input to the experiment. A VCF file was obtained representing the SNPs assigned to each group of genetically distinct cells as determined by freemuxlet. Demuxlet was then run using the reference VCF generated from single-line genotyping. SNPs were then intersected between the VCF file used for demuxlet and the VCF file using a maximum-likelihood approach assigning each unknown freemuxlet cluster to a known reference cell line from the demuxlet VCF file to obtain final cluster assignments by genotype.

Integration of Transcriptome Information with Deconvolution by Genetics

Single-cell RNA sequencing formatted gene count matrix data from mRNA reads were overlaid with single cell genotype calls assigned by deconvolution by genetics. Without knowledge of these single cell genotype calls, over-clustering was carried out at a leiden sparsity factor greater than ten or by assignment of the leiden factor to result in a total number of clusters according to the following formula:

clusters_number=10*number_of_celltypes_in_pool

Using a custom integration function of transcriptome based and genetics based calls, a final list of cell types assigned based on both sources of data was arrived at. For each leiden cluster, the percentage of cells within that cluster belonging to a particular SNP-determined genotype was calculated. If cells from that cluster belonged to greater than 70% one SNP-determined genotype this cluster was marked as high confidence by both genetic and transcriptome methods and genotype assignment for that cluster was changed to the population representing greater than 70% of all the cells. All other clusters with <70% confidence were marked for deletion as low-confidence data points that did not harmonize between both genetics and transcriptome assignments.

### Representative Code for Example Five:

```
format_freemuxvcf:
      ### subset only those that uniquely distinguish between samples
      freemux_uniq = freemux_full.apply(lambda row: is_uniq(row),axis=1)
format_demuxletvcf:
      ### subset only those that uniquely distinguish between samples
      demux_uniq = demux_full[demux_full.index.isin(freemux_uniq.index)]
    match_demux_freemux(demux_uniq,freemux_uniq):
      ###### compare the two snps between freemux and demux
      snpsum =
    compare_snps(demux_uniq.loc[snpid],freemux_uniq.loc[snpid])
      ###### get total possible number of comparisons
        snptotals = get_snptotals(demux_uniq)
      ###### divide by total possible number of comparisons
      tmp_row = [x/snptotals[i] for x in tmp_row]
      print(snptotals[i],tmp_row)
      sums_matrix.append(tmp_row)\
      ###### get final assignment dictionary
        assign_dict = get_assigndict(sums_matrix)
###### Demuxlet assignment to single-cell data
  correct_demuxlet(adata,cutoff=0.7,label=‘cell_type’):
    ##### This function corrects integrates demuxlet and transcriptome calls by
    assigning a cell type to each cluster in transcriptome space. It uses cutoffs as
    defined in parameters which refers to proportion of max cell types.######
      celldict = { }
      ###
      for clust in set(adata.obs.leiden):
      ##get cluster alone first
      clustdf = adata[adata.obs.leiden==clust].obs
      ##calculate proportions
      props = clustdf.groupby(label).count( )[‘barcode’]/(len(clustdf))
      ##see if there is a majority based on cutoff
      majorcell = props[props>cutoff].index.tolist( )
      ##change all cell_type values to the most abundant value
      cells = clustdf.index.tolist( )
      if len(majorcell) == 1:
      ##add to dictionary
      celldict[clust] = majorcell[0]
      else:
      celldict[clust] = ‘delete’
      ###replace cell type calls
      adata.obs[label] = adata.obs.apply(lambda row:
    replace_celltypes(celldict,row,label),axis=1)
      ###remove cells flagged as non confident calls (probable doublets)
      adata = adata[adata.obs[label]!=‘delete’]
```

### Example 6—Identification of Experimental Sample Origin Using Noise-Corrected Algorithms for Sample Hashing Antibodies

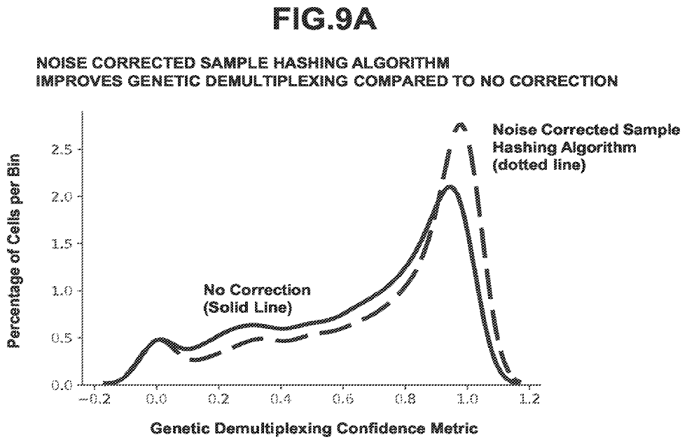

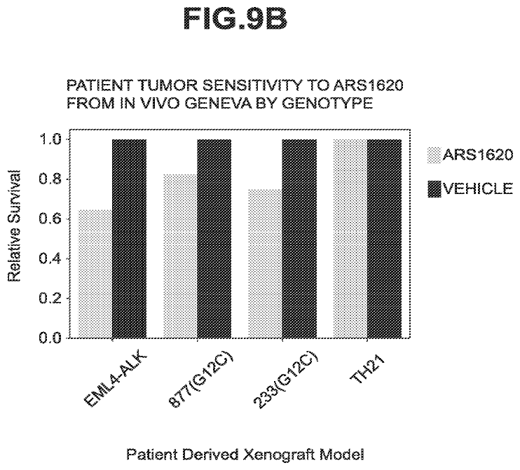

This example is directed at assignment of single cells to their sample of origin using a noise-corrected sample hashing algorithm. Single cells were demultiplexed according to antibody labelling (Totalseq from Biolegend) sub-library data using a custom baseline read-adjusted algorithm. Each cell was mapped to its cell pool of origin (or equivalently, to the drug with which it was treated). A confidence metric associated with each cell assignment was developed and accuracy of sample origin identification improved to greater than 90% and accuracy increased over the standard method of maximum-read assignment (FIG. 9A).

### Custom Baseline Read-Adjustment Algorithm

The steps described here outline the workings of the custom baseline read-adjustment algorithm. Obtained were single-cell RNA sequencing barcode identifiers as a whitelist from the 10× Genomics Cellranger Pipeline. Generated was a table listing the antibody hash sequences and the corresponding samples associated with those hashes. We read in raw fastq data and stripped off constant sequences from raw reads to isolate scRNAseq barcode region alone and antibody labelling barcode region alone. Barcodes were corrected from the scRNAseq barcode reads to the single-cell whitelist within a hamming distance of 1. Barcodes were corrected from the antibody labelling barcode reads to the antibody hash sequences within a hamming distance of 1. Reads were filtered by post-hamming corrected barcodes for absolute match to both whitelists. Barcodes were then assigned by a median base-line read adjustment algorithm. For each antibody hash, background noise to subtract was calculated according to the following formula:

median_correction_factor*median_reads_per_antibody

where the median_correction_factor is typically ˜1.6 for best performance. For each cell the number of reads calculated as background for that specific antibody hash was subtracted to obtain a custom per-antibody denoised dataset of raw reads. For each cell the percentage of reads coming from each antibody hash was calculated. We returned a final list of single cell barcodes and their corresponding highest probability antibody hash and a corresponding confidence interval value. The confidence interval was calculated by taking the percentage of reads coming from the best identifier and subtracting the percentage of reads coming from the second best identifier. The highest scoring antibody hash from each cell was assigned as the best identifier. Cells with confidence intervals<=0.4 were removed.

### Representative Code for Example 6

```
def correct_median(readmatrixmed_factor=1.4):
##### subtract the median multiplied by the factor for each barcode (ie remove background)
  testmed = pivot.transpose( )−med_factor*(pivot.transpose( ).apply(np.median))
##### set negatives to zero
  testmed[testmed<0] = 0
###calculate pct per cell
  testmed = testmed/testmed.sum( )
####get significance value and call cells
  new = test.copy( )
  new[‘call’] = test.apply(lambda row: get_sig(list(row),list(test.columns)),axis=1)
pymulti(R1,R2,bcs10x,len_10x=16,len_umi=12,len_multi=8,med_factor=1.6,gbc_thresh=None,
sampname=‘pymulti_’,split=True,plots=True,hamming=False,thresh=False,pct_only=False,med
ian_only=False,huge=False,bcsmulti=None,reads=None, thresh_dict={ }):
″″″ main loop, splits from fastqs and runs through cell calls
  R1 = your Read1 fastq for the multiseq/hashing fraction
  R2 = your Read2 fastq for the multiseq/hashing fraction
  bcsmulti = your whitelist of known multiseq/hashing barcodes
  bcs10x = your whitelist of known cell identifiers
  len10x = the length of the 10x barcodes
  len_umi = the length of the umis on each pair of reads
  len_multi = the length of the multiseq barcode sequence the package will split out
  med_factor = if doing median correction, this is the factor that is multiplied by the
median to estimate the amount of background reads per barcode
  sampname = this is the handle with which intermediate files will be saved
  split = True if you have not split it from fastq yet, False if you have split it already and
want to rerun the processing. this will save time.
  hamming = True if you want to use match multiseq barcodes within hamming distance
of 1 to the multiseq/hashing whitelist
  thresh = True if you want to use a threshold on which to gate reads, different correction
method
  pct_only = True if you only want to use the raw percentage of reads to call the barcode
  median_only = True if you want to use the median correction method
  huge = True if your file is huge in which case it will be saved as an h5 file instead of
pickling
  thresh_dict = a dictionary of barcodes:threshold which will be used to gate reads on the
samples
  ″″″
  ###
  if huge == True: print(‘assuming huge fastqs.’)
  ###split fastqs and pickle
  os.system(‘mkdir pymulti’)
  if split == True: reads =
split_rawdata(R1,R2,len_10x,len_umi,len_multi,sampname,huge=huge)
  ###read in old pickle data
  readtable = read_pickle(sampname,reads=reads,huge=huge)
  #####check duplication multi and 10x rates
  multirate = check_stats(readtable,bcsmulti,bcs10x)
  ####match by hamming distance
  if hamming == True:
  readtable.drop_duplicates(inplace=True)
  matchby_hamdist(readtable,bcsmulti,bcs10x)
  #####check duplication multi and 10x rates
  multirate = check_stats(readtable,bcsmulti,bcs10x)
  ####filter readtable
  filtd = filter_readtable(readtable,bcsmulti,bcs10x,gbc_thresh)
  ####implement multiseq correction by within distribution zscores
  if median_only == True:
  correct_median(filtd, sampname,med_factor,plots)
  else:
  correct_simple(filtd, sampname,plots,thresh,pct_only,thresh_dict)
  return(filtd)
```

### Example 7—Determination of Genetic Drivers of Sensitivity to a Candidate Agent Using GENEVA

This example is directed at discovery of genetic drivers of sensitivity to a drug compound by way of simultaneous inference of phenotype from a GENEVA cell pool. Long-duration growth balanced cell pools were created and subjected to drug treatment. After assignment to patient of origin and drug treatment condition using genetic demultiplexing using the method as described in Example 5 and sample hashing demultiplexing as described in Example 6, datasets were obtained with discrete numbers of cells in each drug treatment condition as evidenced in FIG. 2A,B,C,D,E,F. The relative number of viable cells remaining in each drug treatment condition for each cell type was counted and this drug sensitivity information was used as the response variable for a linear model. Full exome genetic mutation data for all cell lines was obtained and these features were used as explanatory variables for a linear model regression. A Lasso feature selection model was applied to determine which genetic mutations were responsible for the drug sensitivity to different compounds. Known compounds targeting cell lines with specific genetic mutations were used in proof of concept experiments, specifically Vemurafinib (VEM) which targets cells harboring a BRAF.V600E mutation or ARS-1620 (ARS) which targets cells harboring a KRAS.G12C mutation. Regression using a Lasso Model correctly predicted for both Vemurafinib and ARS-1620 the mutation which predisposes sensitivity to the compound, in line with prior knowledge about these compounds (FIG. 3B, 3C). Using several doses of ARS1620 enough information was produced in one pooled experiment to generate survival curves for multiple cell types (FIG. 3D) equivalent to simultaneous IC50 measurements. These relative sensitivities were compared across cell lines to rank cell lines that were more or less sensitive to ARS1620 (FIG. 3E).

Transcriptomic data from GENEVA assays were also used for determination of drug phenotype. Following drug treatment using ARS-1620 in both in vivo and ex vivo systems, patients derived xenografts with known KRAS.G12C mutations were shown to be sensitive to the compound (patients 877 and 233) by estimation of cell cycle inhibition rates (FIG. 3F). In this PDX context, the dataset also allowed us to infer the sensitive driver mutation (KRAS.G12C).

### Calculation of Fitness of Cell Types Against Drugs

The number of cells in each drug condition were counted according to the origin cell type. These raw numbers were then used as input for a normalization-based function that adjusted for differences in total cell numbers between drug treatment conditions and returned an adjusted fitness calculation for each cell type according to the following formulas: For each cell type, Ci, for each drug and vehicle pair, Dx and D0 respectively, and given a matrix of cell counts with the following data structure:

| | Dx | D0 |
|---|---|---|
| Ci | #cells | #cells |
| Ci + 1 | #cells | #cells |
| Ci + 2 | #cells | #cells |
| . . . | | |

fitness for cell type Ci against Drug Dx based on proportional representation was calculated as:

F(Ci,Dx)=((#cells Dx,Ci)/SUM(#cells Dx))/(#cells D0,Ci)/SUM(#cells DO))

Samples were also adjusted based on the dataset size according to relative sample normalization by calculating an adjustment factor to account for total cell number differences that could escape proportional calculations:

For each drug or vehicle Dx, the total number of cells was counted in each condition, the geometric mean of the total cell counts was calculated, and the total number of cells in each condition was divided by the geometric mean of the cell counts to obtain a normalization ratio. The following matrix was divided by the corresponding normalization for each condition Dx to obtain a final corrected matrix of cell counts adjusted for dataset size according to a geometric mean ratio based correction.

| Dx | D0 | |
|---|---|---|
| Ci | #cells | #cells |
| Ci + 1 | #cells | #cells |
| Ci + 2 | #cells | #cells |
| . . . | | |

For each of these fitness calculations, the relative fitness was calculated as:

Fitness for Cell Line Y=(Fitness for Cell Line Y)/(Maximum Fitness between all Cell Lines within Pool)

### Lasso Regression for Discovery of Genotype Drivers of Drug Sensitivity

Mutations derived from whole exome data sequencing data for each cell type were downloaded and categorized for intersection across a minimum of two different cell types. These were then formatted into a table of dependent variables suitable for input into a lasso regression algorithm for feature selection. In a paired fashion the fitness for each cell line was also calculated and formatted as the response variable for the lasso algorithm. The lasso regression was then run with alphas between 0.05 and 0.30 with 50 interval steps to find the most relevant mutations predictive of GENEVA cellular response. Highest scoring explanatory variable genetic mutations were ranked by their covariates and designated as drivers of drug sensitivity.

### Reconstructed IC50 Curves Based on GENEVA Phenotypes Over ARS-1620 Multiple Dose Curve

Cell counts were obtained across different drug treatment conditions specifically across different concentrations of drugs. At the point of harvest, these numbers were annotated and used to calculate the absolute number of cells of each cell type. The formula was as follows:

Absolute Number of Cells of Cell Type, Cy, in Drug Condition, Dx=(Number of counted cells of Cy in Dx)/(Total number of counted cells in Dx)*Number of cells counted from annotation at harvest

These numbers were then used as the input to a logistic regression fit and IC50 curves were interpolated from the multiple dosing cell counts.

### Determination of Cell Cycle Inhibition Rates as Alternative Proxy for Cell Counting as Phenotypic Readout

Cell Cycle was calculated by regressing whole transcriptome readouts and weighting according to specified genes of interest related to different cell cycle states. Cells were assigned into G1, S, and G2/M. The cell cycle ratio was then calculated as fraction of cells per population:

(#cells in G2M)/(total #cells)

These ratios were then plotted as a linear function across the experimental dose regimen they were subject to, in this case a dose curve of ARS-1620 (uM). The linear fit of this function with:

x=ARS−1620 (uM)

y=(#cells in G2M)/(total #cells)

yielded a function where the slope was determined as the cell cycle inhibition rate, reflecting an alternative way to measure phenotype derived solely from transcriptome drug perturbation data over a long time course.

### Representative Code for Example 7

```
##########
Calculation of Fitness of Cell Types against Drugs
##########
#### get lengths of each treatment dataset
  sizedict={ }
  for drug in set(adata.obs[treatment]):
  onedf = adata[adata.obs[treatment]==drug].obs
  sizedict[drug] = len(onedf)
  #### deseq normalize
  if deseq_norm==True:
  ##count cells by type
      for celltype in set(adata.obs[label]):
  #### count
      onedf = adata[adata.obs[label]==celltype]
      props = pd.DataFrame(onedf.groupby([treatment]).count( )[‘barcode’])
      results[celltype] = props[‘barcode’]
      ##### create pseudo reference and execute deseq
      pseudo = results / gmean(results.iloc[:,:],axis=0)
      ##### correction
      results = results.transpose( )/ratios.tolist( )
      ##### divide everything by the vehicle
      results = results.div(results[vehicle],axis=0)
      ####regular normalization
      else:
      ####calculate relative proportions by cell line to get fitness relative to vehicle
      ##save data to results
      for celltype in set(adata.obs[label]):
      #### count
      onedf = adata[adata.obs[label]==celltype].obs
      props = pd.DataFrame(onedf.groupby([treatment]).count( )[‘barcode’])
props = pd.DataFrame(props.apply(lambda row: row[‘barcode’] /
  sizedict[row.name] , axis=1))
      ###divide everything by the vehicle
      results = results.div(results[vehicle],axis=0)
      ###scale everything to max
      results = results/results.max( )
##########
Lasso Regression for Discovery of Genotype Drivers of Drug Sensitivity
##########
  ####### get only mutations that are present in more than one
  uniq = pd.DataFrame(mutations_table.sum(axis=0))
  uniq = uniq[uniq[0]!=1]
  uniq = uniq.index.tolist( )
  table = table[uniq]
  ##### import response variable
  vem = fitness[‘VEM’]
  ars = fitness[‘ARS1620’]
  ######## put in response variable into gene expression data
  vem_x[‘y’] = vem_x.apply(lambda row: vemurafinib_dict[row.name],axis=1)
  ############ define alpha range to search
  alphas = np.linspace(0.05,0.3,50)
  alphas = alphas[::−1]
  coefs = [ ]
  ######## calculate alphas
  for alpha in alphas:
      reg = linear_model.Lasso(alpha=alpha)
      reg.fit(vem_x, vem_y)
      coefs.append(reg.coef_)
  ######## visualize coefficients for all gene mutations
  log_alphas = −np.log10(alphas)
  coefs = np.transpose(coefs)
  for coef,c in zip(coefs,colors):
  plt.plot(log_alphas,coef,c=np.random.rand(3,),linewidth=3.0)
##########
Reconstructed IC50 Curves based on GENEVA phenotypes over ARS-1620 multiple dose curve
##########
    #### Scale number of cells across cell type and drugs
    tmp = invitro_clean.obs[‘cell_line’,‘sample’]]
    tmp = pd.pivot_table(tmp, values=‘index’, index=[‘cell_line’], columns=[‘sample’],
    aggfunc=lambda x: len(x.unique( )))
    #### Define dose regimens
    conc_dict = {
      ‘C0_1’:0.00,
      ‘C0_2’:0.00,
      ‘C1_1’:0.4,
      ‘C1_2’:0.4,
      ‘C2_1’:1.6,
      ‘C2_2’:1.6,
      ‘C3_2’:6.3,
      ‘C4_1’:25.0,
      ‘C4_2’:25.0
    }
    ##### multiply by number of cells from annotation at harvest
    tmp = tmp*[annotated_actual_cell_counts]
    ##### logistic regression
    sns.Implot(data=tmp,x=“concentration (uM)”,y=″%survival, logistic=True)
```

### Example 8—Identification of a Mitochondrial Mechanism of Action of a Candidate Agent in Long Term Drug Treatment Using GENEVA

This example is directed at identification of the molecular mechanism of action of the compound ARS1620 by way of mitochondria gene down regulation using GENEVA.

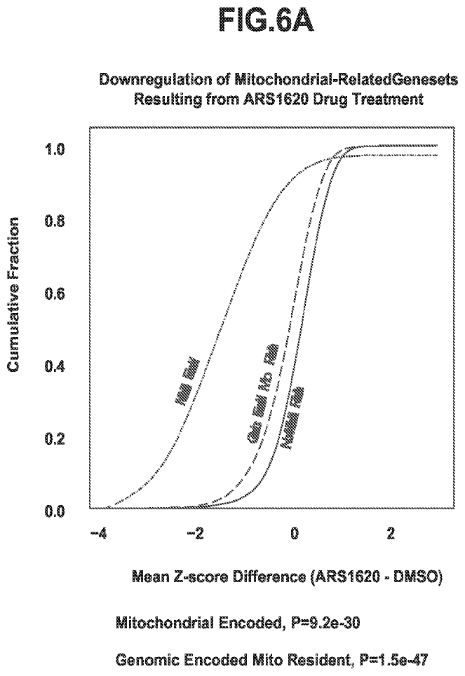

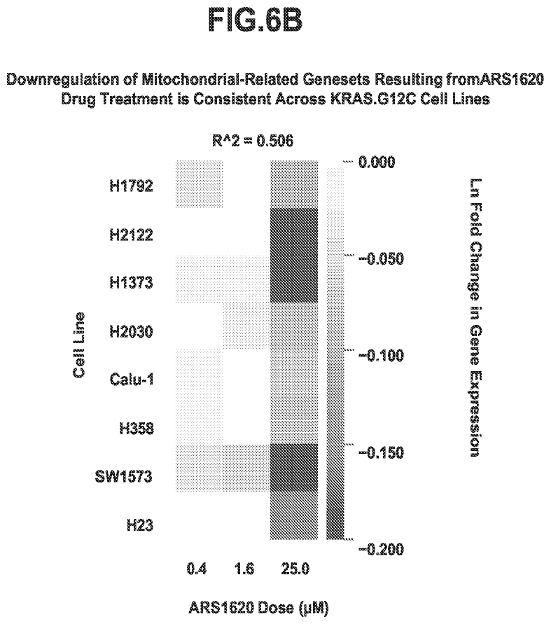

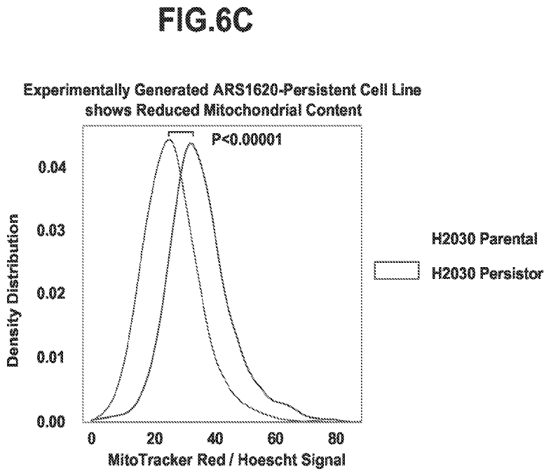

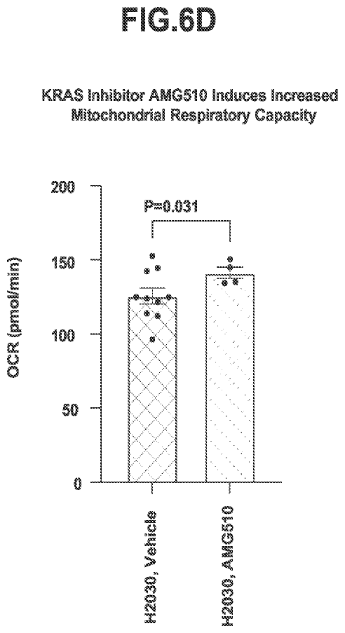

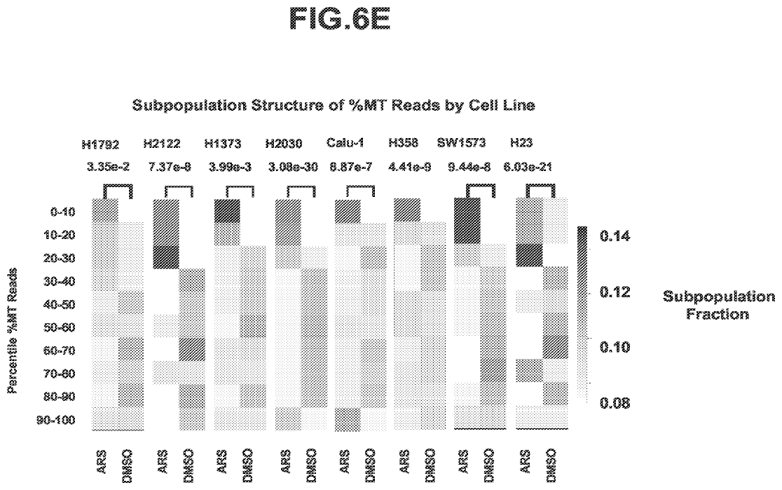

Long-time course GENEVA pools were created to understand how ARS1620 achieved durable tumor regression in human cells. A seven day drug treatment regimen was conducted and transcriptome changes in surviving cells were analyzed. Mitochondrially encoded genes were downregulated indicating an effect of ARS1620 on the mitochondria (FIG. 6A-B). Generation of an experimental line that had long term treatment under ARS1620 as validation showed that mitochondria are significantly ablated after long term ARS1620 treatment (FIG. 6C). Using an assay of cellular respiration, a significant difference in functional oxygen consumption at the site of the mitochondria was also detected as a result of ARS1620 treatment (FIG. 6D). An analysis of subpopulation composition of cells within each cell line comprising the GENEVA pool revealed that after drug treatment with ARS1620 cells that survived showed significantly lower percentage of mitochondrial reads relative to overall reads indicating a population level selection pressure for cells with downregulated mitochondrial gene expression (FIG. 6E).

### Differential Expression for Each Cell Line Between Drug and Non-Drug Conditions for Causal Geneset Discovery

For each cell line, single cell datasets were divided into two sub-datasets: vehicle treated and drug treated. Differential expression was calculated between two single-cell datasets using a two-sample t-test. A differential expression score was obtained for each gene from two-sample t-test output. All cell lines were repeated until done and all differential expression scores for genes were saved into aggregated differential expression tables by cell line. Cells were grouped according to their phenotypic sensitivity to the tested compound. For tested compounds, the genetic driver of the compound sensitivity was discovered as described previously herein. Using the genetic driver, cell lines were classified by presence or absence of the driver into two categories, sensitive vs non-sensitive. The differential expression matrix was grouped into sensitive and insensitive cell lines. z-scores for genes within each cell line were calculated, genes were grouped by gene sets from biological geneset databases curated from scientific literature, and two sample T-tests with grouped z-scores were performed. First, all differential expression scores were normalized to the same scale by z-score within each cell line. For all genes within the differential expression matrix created a dictionary of groupings taken from each of the mSIGDB databases (https://www.gsea-msigdb.org/gsea/msigdb/). For each grouping of genes from mSIGDB, two sample T-tests were performed for each gene set individually treating all “sensitive” and “insensitive” cell lines as replicates. Summary statistics from each gene test were saved and the z-score difference was calculated. The biological mechanism of action of the molecule of interest was determined by rank ordering the median z score results from each geneset to determine the relative upregulation or downregulation of genesets in response to the compound.

### Representative Code for Example 8

```
#### de score matrix
de_score_all_lines
#### msigdb full database
msigdb
####
de_zscored_all_lines = de_score_all_lines.apply(zscore,axis=0)
genesets_zscored = de_zscored_all_lines.groupby(by=msigdb)
ttest_results = genesets_zscored.apply(lambda row: stats.ttest_ind( row[′sensitive’],
row[‘insensitive’] ) ,axis=1)
median_zscore_results = genesets_zscored.apply(lambda row: np.median(row[′sensitive’]) −
np.median(row[‘insensitive’]) ,axis=1)
#### these are identities of sensitive and insensitive lines categorized by genetic driver
sensitive_lines
insensitive_lines
#### de score matrix
de_score_all_lines
####
For cell_line in de_score_all_lines:
  if cell_line in sensitive_lines:
    de_score_all_lines[cell_line].index.replace(“sensitive”)
if cell_line in insensitive_lines:
  de_score_all_lines[cell_line].index.replace(“insensitive”)
### scdata contains all single-cell data in anndata data structure format
### de_score_all_lines contains final output from this section
Import scanpy as sc
For cell_line in all_lines:
  vehicle_data = scdata[scdata.obs.drug == ‘vehicle’]
  drug_data = scdata[scdata.obs.drug == ‘drug’]
  differential_expression = sc.tl.rank_genes_groups(vehicle_data, drug_data, method=’t-
test′)
  de_score = scdata.uns[‘t-test’][‘scores']
  de_score_all_lines.append(de_score)
```

### Example 9—Identification of Mechanism of Action of a Candidate Agent in Inducing Ferroptosis Using GENEVA

This example is directed at identification of the molecular mechanism of action of the compound ARS1620 by way of ferritin gene up-regulation using a GENEVA cell pool.

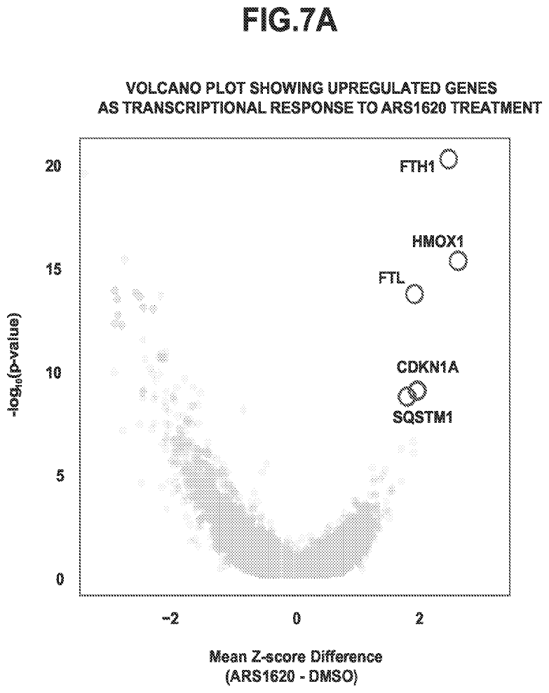

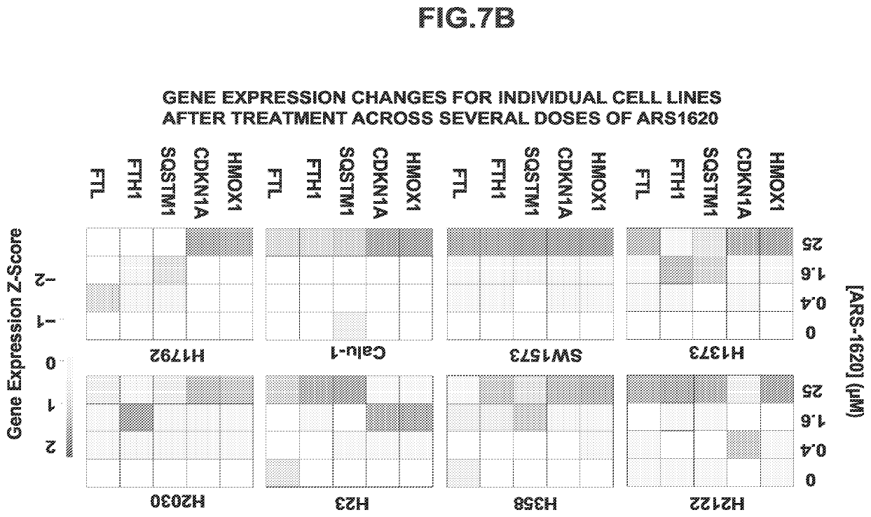

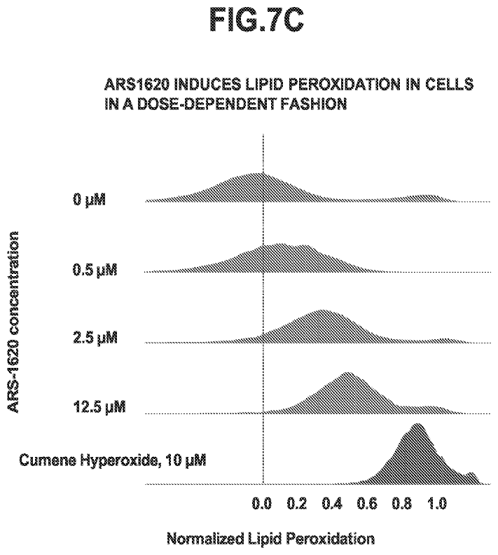

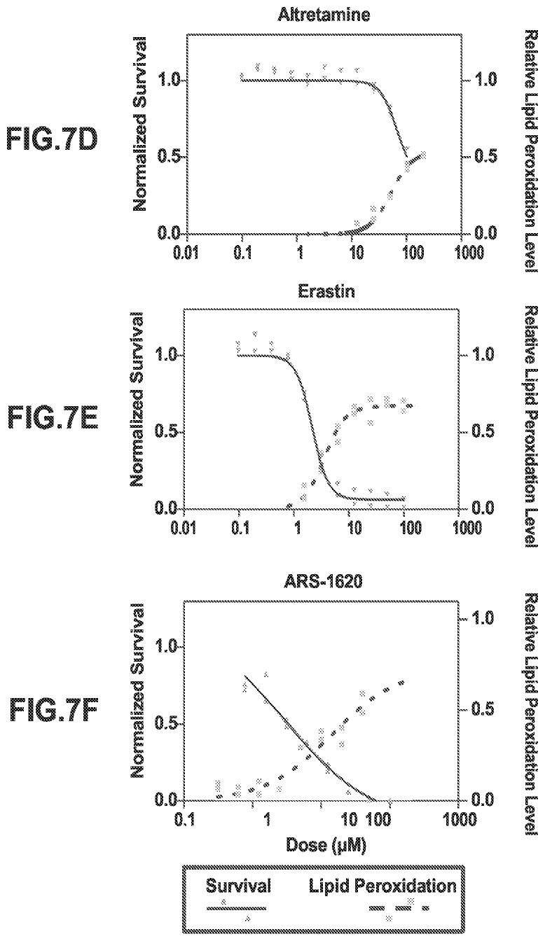

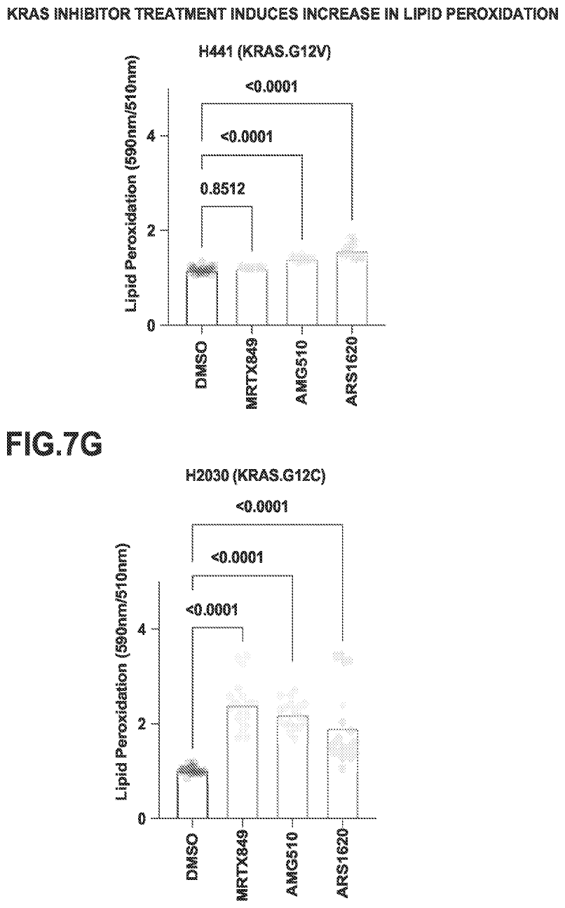

Upregulated genes were analyzed across KRAS.G12C lines in the cell pool in cells surviving long-term ARS1620 treatment. Consistently upregulated genes across cell lines were found to be involved in an anti-ferroptotic response mechanism (FIG. 7A,B). Among this group of genes were FTH1 and FTL, the two components of the Ferritin Complex, which is responsible for sequestration of labile free iron. Using a lipid peroxidation live cell probe, lipid peroxidation—one of the hallmark phenotypes of ferroptosis—was measured in response to ARS-1620 treatment. A dose curve demonstrated that ARS-1620 induced lipid peroxidation in a dose dependent fashion (FIG. 7C). Furthermore, comparison of cell survival and lipid peroxidation dynamics with known inducers of ferroptosis (Erastin and Altretamine) showed that ARS-1620 behaves similarly in its ferroptotic/survival kinetics to these ferroptosis inducers (FIG. 7D). Survival and normalized lipid peroxidation curves crossed over around the IC50 survival value in all three compounds demonstrating similar pharmacodynamics. In a KRAS.G12C line (H2030) all three KRAS.G12C inhibitors MRTX849, AMG510, and ARS1620 increased lipid peroxidation while in a non-KRAS.G12C line (H441) this effect was significantly muted (FIG. 7E).

### Example 10—Identification of Drug Resistance Mechanisms Using GENEVA

This example is directed at identifying multiple targetable drug resistance mechanisms from a long time course drug treatment in GENEVA cell pools.

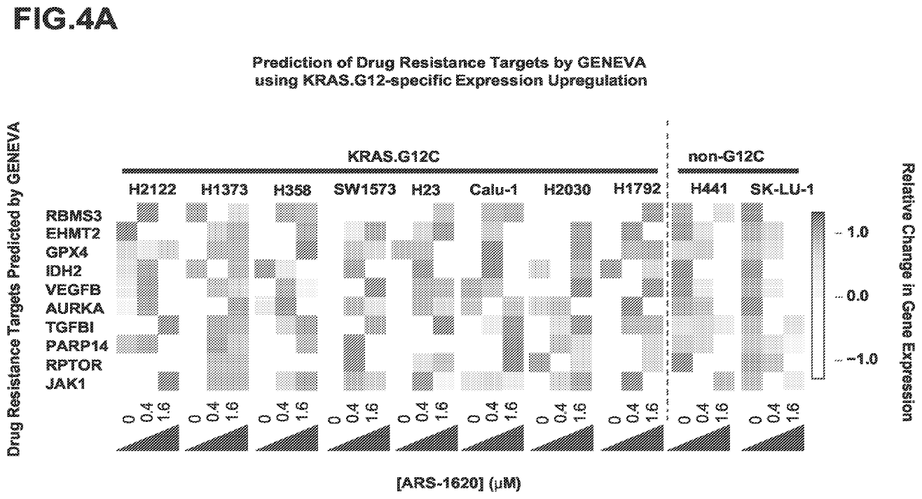

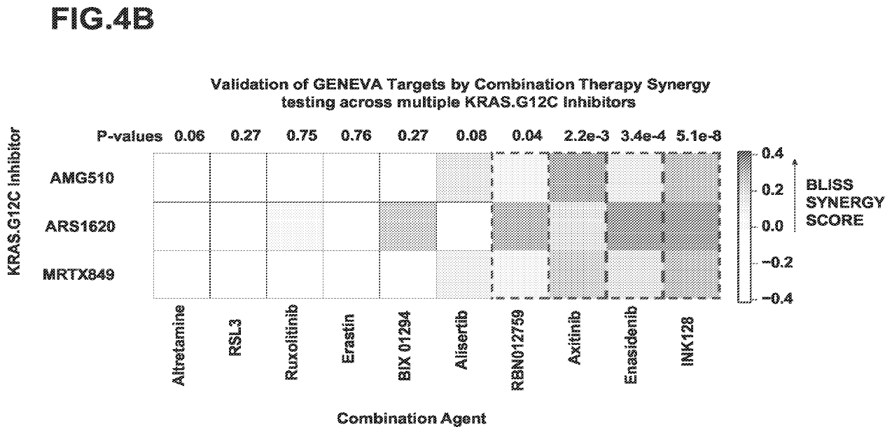

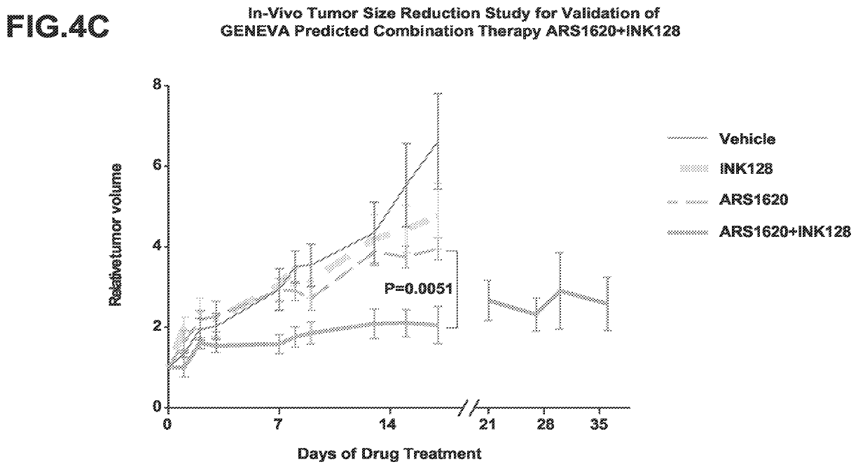

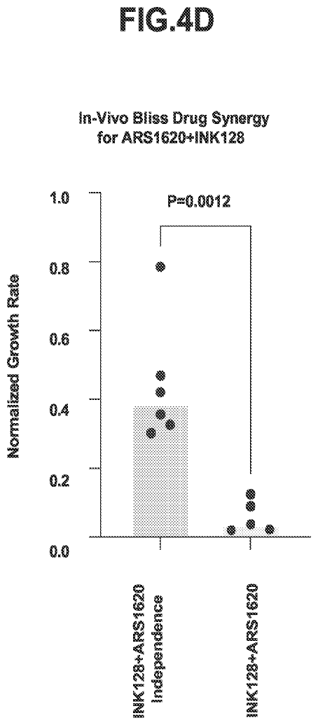

Using the GENEVA dataset of ARS-1620 in a fourteen day drug treatment timeline in which drug resistance developed, gene expression indicators of resistance mechanisms were obtained (FIG. 4A). Inhibitors of these targets were obtained and tested for drug synergy in combination with three KRAS.G12C inhibitors, ARS1620, AMG510, and MRTX849; where drug synergy is defined as the sum of the drug combination being greater than an additive model of the individual compounds alone. Several GENEVA predicted drug targets demonstrated high Bliss synergy scores (scores greater than zero indicated significant drug synergy, FIG. 4B). The strongest GENEVA prediction, mTOR resistance, was tested in a multi-arm in vivo mouse study and significant in vivo Bliss drug synergy and tumor reduction over time was found (FIG. 4C,D).

### Differential Expression for Each Cell Line Between Drug and Non-Drug Conditions for Identification of Drug Resistance Pathways

For each cell line, the single cell dataset was divided into two sub-datasets: vehicle treated and drug treated. Differential expression was calculated between two single-cell datasets using a two-sample t-test. A differential expression score was obtained for each gene from two-sample t-test output. This was repeated with all cell lines until done and all differential expression scores for genes were saved into aggregated differential expression tables by cell line. z-scores were calculated for genes within each cell line and two sample T-tests with grouped z-scores were performed. All differential expression scores were first normalized to the same scale by z-score within each cell line. Two sample T-tests were then performed for each gene individually treating all “sensitive” and “insensitive” cell lines as replicates. Summary statistics were saved from each gene test and calculated the z-score difference. Genes were subsetted by summary statistics and druggability of gene products. Using t-test results subsetted by p-value for each gene by selecting genes with p-values less than or equal to 0.01. Using median results subsetted by z score change delta between sensitive and insensitive lines for each gene by selecting genes with z score values greater than the top 80th percentile of gene scores. Using DGIDB.org, cross-referenced genes obtained from summary statistics with druggable targets from DGIDB.org as a resource of druggable targets and obtain final target lists for drug resistance targets induced by compounds of interest.

### Representative Code for Example 10

```
### scdata contains all single-cell data in anndata data structure format
### de_score_all_lines contains final output from this section
Import scanpy as sc
For cell_line in all_lines:
  vehicle_data = scdata[scdata.obs.drug == ‘vehicle’]
  drug_data = scdata[scdata.obs.drug == ‘drug’]
  differential_expression = sc.tl.rank_genes_groups(vehicle_data, drug_data, method=’t-
test′)
  de_score = scdata.uns[‘t-test’][‘scores']
  de_score_all_lines.append(de_score)
#### de score matrix
de_score_all_lines
####
de_zscored_all_lines = de_score_all_lines.apply(zscore,axis=0)
ttest_results = de_zscored_all_lines.apply(lambda row: stats.ttest_ind( row[′sensitive’],
row[‘insensitive’] ) ,axis=1)
median_zscore_results = de_zscored_all_lines.apply(lambda row:
np.median(row[′sensitive’]) − np.median(row[‘insensitive’]) ,axis=1)
#### these are identities of sensitive and insensitive lines categorized by genetic driver
sensitive_lines
insensitive_lines
#### de score matrix
de_score_all_lines
####
For cell_line in de_score_all_lines:
  if cell_line in sensitive_lines:
      de_score_all_lines[cell_line].index.replace(“sensitive”)
if cell_line in insensitive_lines:
  de_score_all_lines[cell_line].index.replace(“insensitive”)
    ttest_results
median_zscore_results
####
ttest_filtered = ttest_results[ttest_results[‘pvalue’]<=0.01]
    median_zscore_filtered =median_zscore_results[median_zscore_results[‘zscore’]>=
    np.percentile(median_zscore_results[‘zscore’],80)]
    intermediate_genelist
    =set(ttest_filtered[′genes]).intersect(set(median_zscore_filtered[‘genes']))
#### intersect final list with DGIDB
DGIDB_genelist
final_genelist = set(intermediate_genelist).intersect(set(DGIDB_genelist))
```

### Example 11—Identification of In-Vivo-Specific Drug Resistance Mechanisms Using GENEVA

This example is directed at identification of induction of the Endothelial-Mesenchymal Transition as an in-vivo specific mechanism of tumor resistance to ARS1620 using GENEVA.

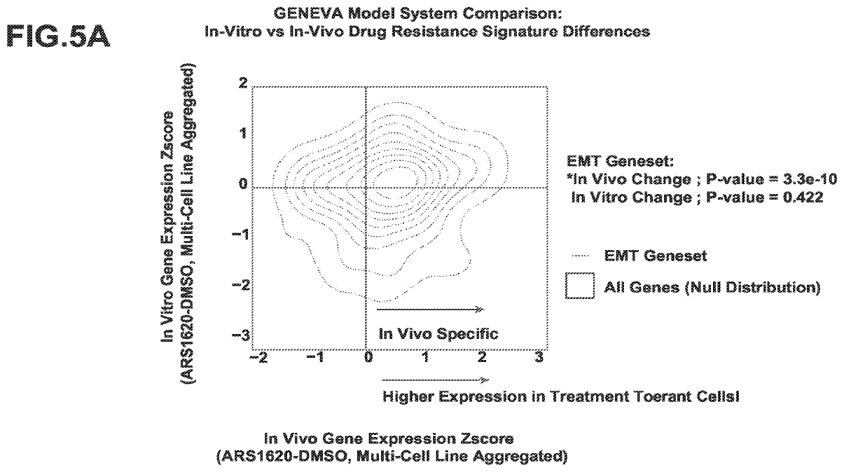

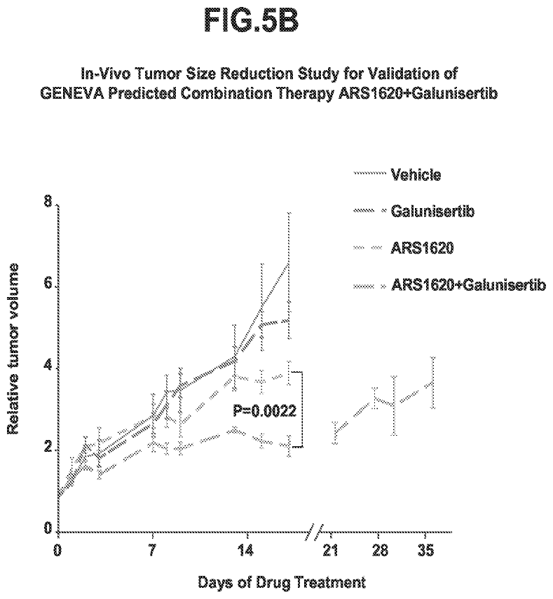

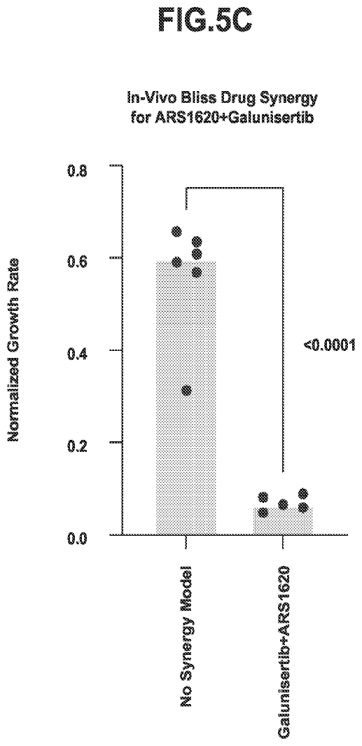

The GENEVA datasets were compared where GENEVA pools were drugged with the KRAS.G12C inhibitor ARS1620, conducted both in vitro and in vivo. Specifically, the in vitro data were compared against the in vivo data to look for differences and similarities attributable to the context of the model systems used. One of the most upregulated gene sets in response to drug was specific to the in vivo context and showed no difference in vitro (FIG. 5A). The endothelial-mesenchymal transition hallmark gene expression signature was increased in vivo and represented a possible drug-adaptive mechanism to ARS1620 KRAS.G12C inhibition. As a validation study, a combination therapy multi-arm in vivo mouse study was designed to test the efficacy of an EMT inhibitor, Galunisertib in combination with ARS1620. The combination therapy was found extremely effective in suppressing tumor growth (FIG. 5B) and acted with ARS1620 to reduce growth synergistically (FIG. 5C).

### Discovery of Causal Genesets Specific to In-Vivo Model Systems

For in vivo and in vitro datasets, we combined the median z score results determined in Example 8 and t-test results, respectively, were combined on a paired geneset by geneset basis. The combined zscore results table from both in vivo and in vitro was then used to calculate an “in vivo Specificity Score” as follows:

For each geneset, “in vivo Specificity Score”=[(in vivo median zscore)−(in vitro median zscore)]/mean([log 10 (in vivo pvalue), log 10 (in vitro pvalue)]

Ordered genesets were then ranked by their in vivo Specificity Score to arrive at genesets significantly upregulated or downregulated specifically in vivo in response to ARS1620.

### Example 12—Testing Efficacy of Combination Therapies Using GENEVA

This example is directed at testing combination therapies to identify an optimal combination therapy in a GENEVA cell pool.

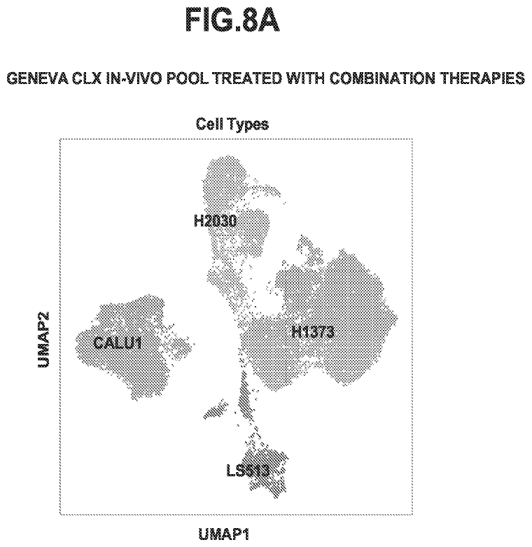

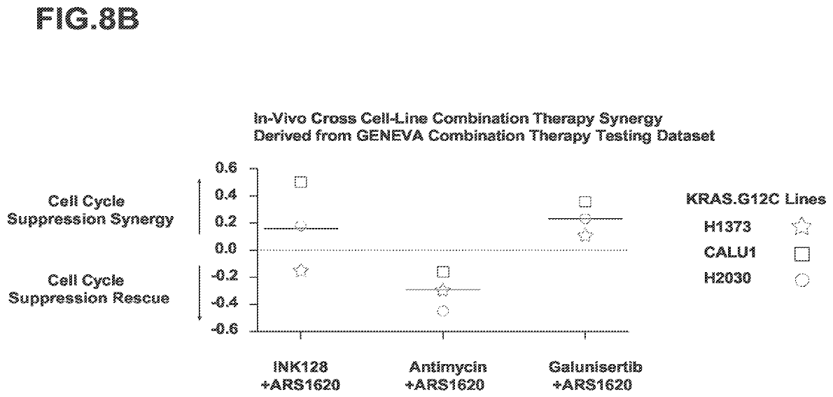

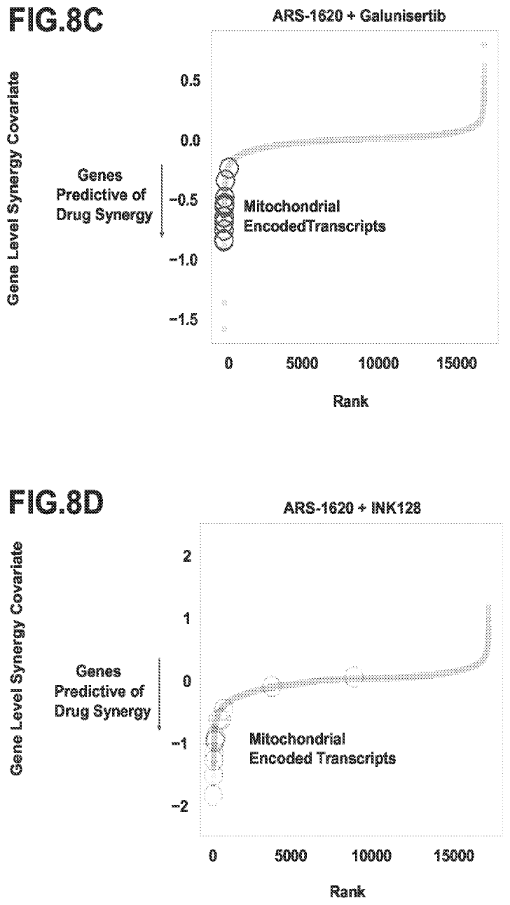

A panel of G12C and non G12C lines pooled in vivo as xenografts was utilized to conduct combination therapy studies comparing ARS1620 with and without Galunisertib, INK128, and Antimycin for a total of eight different treatment conditions; these compounds were taken from our combination therapy discoveries and mechanistic understanding of mitochondrial action of ARS1620 (FIG. 8A). Using cell cycle active/inactive proportions in each drug condition, drug synergy was calculated across several cell lines for each drug. Ink128 and Galunisertib showed relative in vivo synergy consistent with the prior validation experiments demonstrating in vivo synergy, while antimycin showed an antagonistic effect demonstrating a relative rescue of ARS1620 consistent with antagonism of the mitochondrial lethality phenotype (FIG. 8B). A linear model built around gene expression and drug treatment+ cell line of origin to discover genes that drove synergistic drug phenotype revealed that together Galunisertib and INK128 was able to further increase the synergistic decrease in mitochondrial reads consistent with ARS1620 general effect on mitochondria observed alone (FIG. 8C).

### Example 13—Identification of a Patient Subpopulation Sensitive to a Candidate Agent Using GENEVA

This example is directed at detection of a novel patient subpopulation sensitive to a candidate agent (ARS1620) in PDX models.

GENEVA pools of PDX models were created for a long term drug treatment assay. Tumors were implanted in vivo and in organoids, and tumors from KRAS.G12C, EML4-ALK, and TH21 lung cancer patients were drugged in GENEVA pools. Significant drug sensitivity was found in EML4-ALK patient greater than sensitivity of KRAS.G12C mutant tumors indicating EML4-ALK patient tumors would respond to a KRAS.G12C inhibitor (FIG. 9B).

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method for assessing one or more therapeutic properties of a small molecule compound, comprising:
growing a pool of cells of different cell types in three dimensions;
treating the three dimensional pool with the small molecule compound;
dissociating cells of the treated three dimensional pool into single cells;
performing single cell ribonucleic acid (RNA) sequencing on the dissociated single cells and dissociated single cells from a control three dimensional pool not treated with the small molecule compound;
deconvoluting the data from the single cell RNA sequencing into single cell transcriptomes categorized by treatment and cell type; and
assessing one or more therapeutic properties of the small molecule compound based on the categorized single cell transcriptomes.

2. The method according to claim 1, wherein the pool of cells is a growth balanced pool of cells;
wherein
a) each of the different cell types is represented with at least 1×10³ viable cells in the pool of cells;
b) no cell type of the different cell types outnumbers other cell types by 2 orders of magnitude or more in the pool of cells; or
c) the total number of cells of each of the different cell types is within 2 orders of magnitude of each other in the pool of cells.

3. The method according to claim 1 or 2, wherein the pool of cells of different cell types comprises from 2 to 100 different cell types.

4. The method according to claim 1 or 2, wherein the pool of cells of different cell types comprises from 10 to 50 different cell types.

5. The method according to any one of claims 1 to 4, wherein the different cell types comprise primary cells obtained from a patient, cells from an organ system, cells from a disease model, or any combination thereof.

6. The method according to any one of claims 1 to 5, wherein growing the pool in three dimensions comprises producing a xenograft from the pool.

7. The method according to any one of claims 1 to 5, wherein growing the pool in three dimensions comprises producing an organoid from the pool.

8. The method according to any one of claims 1 to 7, wherein the one or more therapeutic properties comprise candidacy of the small molecule compound for combination therapy with a drug, wherein the method comprises, based on the single cell transcriptomes categorized by treatment and cell type:
determining drug sensitivity for each cell line by counting the number of cells remaining in each condition;
calculating drug-induced gene expression changes for each cell line;
assigning a weighted score for each gene based on its predicted relevance to drug sensitivity based on the calculated drug-induced gene expression changes for each cell line; and
predicting combination therapy targets based on the genes having weighted scores above a false discovery rate, wherein genes anti-correlated to drug sensitivity predict drug resistance and therefore represent candidate targets for combinatorial targeting.

9. The method according to any one of claims 1 to 8, wherein the one or more therapeutic properties comprise mechanism of action of the small molecule compound, wherein the method comprises, based on the single cell transcriptomes categorized by treatment and cell type:
determining drug sensitivity for each cell line by counting the number of cells remaining in each condition;
determining drug-induced gene expression changes for each cell line;
aggregating the determined drug-induced gene expression changes across drug-sensitive cell lines;
assigning a weighted score for each gene based on its predicted relevance to drug sensitivity based on the aggregated calculated drug-induced gene expression changes;
identifying genes correlated with aggregated drug sensitivity as those having weighted scores above a false discovery rate; and
predicting mechanism of action of the compound based on the genes correlated with the aggregated drug sensitivity.

10. The method according to any one of claims 1 to 9, wherein the one or more therapeutic properties comprise candidacy of the small molecule compound for treatment of a disease subtype, wherein the method comprises, based on the single cell transcriptomes categorized by treatment and cell type:
determining drug sensitivity for each cell line by counting the number of cells remaining in each condition, wherein each cell line is categorized by its genetic mutations and/or transcriptome signature;
aggregating the determined drug sensitivity across cell lines;
assigning a score for each mutation and/or transcriptome signature that predicts relevance to aggregated drug sensitivity using a variable selection regression algorithm; and
predicting efficacy of the compound in a disease subtype based on the disease subtype having a score above a false discovery rate.

11. The method according to claim 10, wherein the variable selection regression algorithm is a weighted lasso regression algorithm.

12. One or more non-transitory computer-readable media comprising instructions stored thereon, which when executed by one or more processors, cause the one or more processors to:
deconvolute single cell RNA sequencing data into single cell transcriptomes categorized by treatment and cell type, wherein the single cell RNA sequencing data was produced by performing single cell RNA sequencing on dissociated single cells from a three dimensional pool of different cell types treated with a small molecule compound and dissociated single cells from a control three dimensional pool of different cell types not treated with the small molecule compound; and
assess one or more therapeutic properties of the small molecule compound based on the categorized single cell transcriptomes.

13. The one or more non-transitory computer-readable media of claim 12, wherein the one or more therapeutic properties comprise candidacy of the small molecule compound for combination therapy with a drug, and when executed by the one or more processors, the instructions further cause the one or more processors to, based on the single cell transcriptomes categorized by treatment and cell type:
calculate drug-induced gene expression changes for each cell line;
assign a weighted score for each gene based on its predicted relevance to drug sensitivity based on the calculated drug-induced gene expression changes for each cell line; and
predict combination therapy targets based on the genes having weighted scores above a false discovery rate, wherein genes anti-correlated to drug sensitivity predict drug resistance and therefore represent candidate targets for combinatorial targeting.

14. The one or more non-transitory computer-readable media of claim 12 or claim 13, wherein the one or more therapeutic properties comprise mechanism of action of the small molecule compound, and when executed by the one or more processors, the instructions further cause the one or more processors to, based on the single cell transcriptomes categorized by treatment and cell type:
determine drug-induced gene expression changes for each cell line;
aggregate the determined drug-induced gene expression changes across drug-sensitive cell lines;
assign a weighted score for each gene based on its predicted relevance to drug sensitivity based on the aggregated calculated drug-induced gene expression changes;
identify genes correlated with aggregated drug sensitivity as those having weighted scores above a false discovery rate; and
predict mechanism of action of the compound based on the genes correlated with the aggregated drug sensitivity.

15. The one or more non-transitory computer-readable media of any one of claims 12 to 14, wherein the one or more therapeutic properties comprise candidacy of the small molecule compound for treatment of a disease subtype, and when executed by the one or more processors, the instructions further cause the one or more processors to, based on the single cell transcriptomes categorized by treatment and cell type:
aggregate drug sensitivity across cell lines, wherein drug sensitivity is determined for each cell line by counting the number of cells remaining in each condition, wherein each cell line is categorized by its genetic mutations and/or transcriptome signature;
assign a score for each mutation and/or transcriptome signature that predicts relevance to aggregated drug sensitivity using a variable selection regression algorithm; and
predict efficacy of the compound in a disease subtype based on the disease subtype having a score above a false discovery rate.

16. The one or more non-transitory computer-readable media of claim 15, wherein the variable selection regression algorithm is a weighted lasso regression algorithm.

17. A system for assessing one or more therapeutic properties of a small molecule compound, the system comprising:
one or more processors; and
one or more non-transitory computer-readable media comprising instructions stored thereon, which when executed by the one or more processors, cause the one or more processors to:
deconvolute single cell RNA sequencing data into single cell transcriptomes categorized by treatment and cell type, wherein the single cell RNA sequencing data was produced by performing single cell RNA sequencing on dissociated single cells from a three dimensional pool of different cell types treated with a small molecule compound and dissociated single cells from a control three dimensional pool of different cell types not treated with the small molecule compound; and
assess one or more therapeutic properties of the small molecule compound based on the categorized single cell transcriptomes.

18. The system of claim 17, wherein the one or more therapeutic properties comprise candidacy of the small molecule compound for combination therapy with a drug, and when executed by the one or more processors, the instructions further cause the one or more processors to, based on the single cell transcriptomes categorized by treatment and cell type:
calculate drug-induced gene expression changes for each cell line;
assign a weighted score for each gene based on its predicted relevance to drug sensitivity based on the calculated drug-induced gene expression changes for each cell line; and
predict combination therapy targets based on the genes having weighted scores above a false discovery rate, wherein genes anti-correlated to drug sensitivity predict drug resistance and therefore represent candidate targets for combinatorial targeting.

19. The system of claim 17 or claim 18, wherein the one or more therapeutic properties comprise mechanism of action of the small molecule compound, and when executed by the one or more processors, the instructions further cause the one or more processors to, based on the single cell transcriptomes categorized by treatment and cell type:
determine drug-induced gene expression changes for each cell line;
aggregate the determined drug-induced gene expression changes across drug-sensitive cell lines;
assign a weighted score for each gene based on its predicted relevance to drug sensitivity based on the aggregated calculated drug-induced gene expression changes;
identify genes correlated with aggregated drug sensitivity as those having weighted scores above a false discovery rate; and
predict mechanism of action of the compound based on the genes correlated with the aggregated drug sensitivity.

20. The system of any one of claims 17 to 19, wherein the one or more therapeutic properties comprise candidacy of the small molecule compound for treatment of a disease subtype, and when executed by the one or more processors, the instructions further cause the one or more processors to, based on the single cell transcriptomes categorized by treatment and cell type:
aggregate drug sensitivity across cell lines, wherein drug sensitivity is determined for each cell line by counting the number of cells remaining in each condition, wherein each cell line is categorized by its genetic mutations and/or transcriptome signature;
assign a score for each mutation and/or transcriptome signature that predicts relevance to aggregated drug sensitivity using a variable selection regression algorithm; and
predict efficacy of the compound in a disease subtype based on the disease subtype having a score above a false discovery rate.

21. The system of claim 20, wherein the variable selection regression algorithm is a weighted lasso regression algorithm.

22. A balanced cell count culture comprising two or more different cell types that has been cultured for a time period wherein each of the two or more different cell types has a growth rate and wherein each cell type of the two or more different cell types are combined at a ratio inverse to the growth rate of each of the cell type of the two or more different cell types prior to culturing.

23. The balanced cell count culture of claim 22, wherein the time period is from 6 hours to 45 days, from 12 hours to 30 days, from 24 hours to 20 days, or from 72 hours to 14 days.

24. The balanced cell count culture of 22 or 23, wherein,
(i) each of the two different cell types is represented with at least 1×10³ viable cells in the balanced cell count culture;
(ii) no cell type of the at least two different cell types in the balanced cell count culture outnumbers other cell types by 2 orders of magnitude or more; or
(iii) in the balanced cell count culture, the total number of each cell type of the at least two or more different cell types is within 2 orders of magnitude of each other.

25. A balanced cell count culture comprising at least two or more different cell types, wherein a sample of from 0.2% to 10% by volume of the balanced cell count culture comprises at least 500 cells of each of the different cell types, wherein the sample is taken from the balanced cell count culture after the balanced cell count culture is cultured for a time period between 72 hours and 45 days after two or more cell types are combined to create a cell pool and inoculated in a culture media to obtain the balanced cell count culture.

26. A balanced cell count culture comprising at least two or more different cell types, wherein each of the cell types is represented with at least 1×10³ cells in the culture and wherein at least two of the cell types are derived from different cancer tissues.

27. A balanced cell count culture comprising at least two or more different cell types wherein each of the cell types is represented with at least 1×10³ cells in the culture and wherein at least two of the cell types include cancer mutations that are different from each other.

28. The balanced cell count culture of any one of claims 22-27, wherein the balanced cell count culture comprises from 2 to 100 different cell types.

29. The balanced cell count culture of claim 28, wherein the balanced cell count culture comprises from 10 to 50 different cell types.

30. A balanced cell count culture according to any one of claims 22-29, wherein the different cell types comprise cells with cancer mutations, cancer cells from one or more subjects, primary cells from one or more subjects, cells from an organ system, cells from a disease model, cells from a variety of cell lines or any combination thereof.

31. The balanced cell count culture of any one of claims 22-30, wherein the balanced cell count culture is implanted in a model system.

32. The balanced cell count culture of claim 31, wherein the model system is in-vitro model system, an in-vivo model system, or an ex-vivo model system.

33. A method of preparing a balanced cell count culture with at least two or more different cell types, the method comprising:
(a) determining the growth rates for each cell type of the two or more different cell types;
(b) combining the two or more different cell types to create a cell pool, wherein the initial cell count of each of the cell types of the two or more different cell types added to the cell pool is determined based upon the growth rates of step (a); and
(c) culturing the cell pool of step (b) over a time period to create the balanced cell count culture, wherein a sample of from 0.2% to 10% by volume of the balanced cell count culture comprises at least 500 cells of each cell type of the two or more different cell types.

34. The method according to claim 33, wherein the sample of step (c) comprises between 5,000-200,000 cells.

35. The method according to claim 33 or 34, wherein no less than 500 viable cells of each cell type of the two or more different cell types are present in the sample of step (c).

36. The method according to any one of claims 33-35, wherein the representation from each cell type of the two or more different cell types after addition to the cell pool in step (b) is inversely proportional to the cell growth rate of that cell type as determined in step (a).

37. The method according to any one of claims 33-36, wherein the balanced cell count culture comprises between 2-100 different cell types.

38. The method according to claim 37, wherein the balanced cell count culture comprises between 2-50 different cell types.

39. The method according to any one of claims 33-38, wherein the determining the growth rate of step (a) comprises measuring the growth rates for each cell type of the two or more different cell types.

40. The method according to any one of claims 33-39, wherein the different cell types are selected from cells with cancer mutations, cancer cells from one or more subjects, primary cells from one or more subjects, cells from an organ system, cells from a disease model, cells from a variety of cell lines or any combination thereof.

41. The method according to claim 40, wherein the different cell types are selected from one or more xenograft models.

42. The method according to claim 41, wherein the xenografts are derived from one of subjects having a disease.

43. The method according to claim 42, wherein the disease is a neoplastic disease.

44. The method according to claim 43, wherein the neoplastic disease is a cancer selected from one or more of the cancer of head, neck, lung, skin, breast, blood, lymph, bone, soft tissue, brain, eye, reproductive system, circulatory system, digestive system, endocrine system, nervous systems, and of urinary system.

45. The method according to any one of claims 33-44, wherein the method further comprises excluding a cell type in step (b) when the cell type has a growth rate 0.2 fold per day.

46. The method according to any one of claims 33-45, wherein the time period in step (c) is from six hours to 45 days.

47. The method according to claim 46, wherein the time period in step (c) is from 12 hours to 20 days.

48. The method according to claim 47, wherein the time period in step (c) is from 24 hours to 14 days.

49. The method according to any of claims 46-48, wherein the time period in step (c) is 72 hours.

50. The method according to any of claims 46-48, wherein the time period in step (c) is seven days.

51. The method according to any one of claims 33-50, wherein the sample of step (c) is taken at the end of the time period.

52. The method according to any one of claims 33-51, wherein at least two or more samples of step (c) are taken at different time points during the time period.

53. The method according to any one of claims 33-52, wherein the growth rate of each cell type in step (a) are determined by a Calcein-AM growth assay individually or Cell Titer Glo growth assay.

54. The method according to any one of claims 33-53, wherein each cell type of the two or more different cell types are combined at step (b) at a ratio inverse to the growth rate of each of the cell types as determined by the Calcein-AM growth assay and ii) scaled to the total number of days for growth.

55. The method according to any one of claims 33-54, wherein the sample of step (c) is from 0.5% to 3% by volume of the balanced cell count culture.

56. A method of correlating cells from the sample of step (c) of any one of claims 33-55 with the two or more cells of the cell pool of step (b) from the sample of step (c), performing steps further comprising:
(i) performing single cell RNA sequencing on one or more cells from the sample to identify single nucleotide polymorphisms in the one or more cells from the sample; and
(ii) comparing the single nucleotide polymorphisms of step (i) with single nucleotide polymorphisms of the two or more cells of the cell pool in step (b) thereby correlating cells from the sample of step (c) with the two or more cells of the cell pool of step (b).

57. The method according to claim 56, further comprising single cell transcriptome analysis on one or more cells of the sample.

58. The method according to any one of claims 33-57, wherein a portion of step (c) is performed in-vitro.

59. The method according to any one of claims 33-58, wherein a portion of step (c) is performed in-vivo.

60. The method according to any one of claims 33-58, further comprising implanting the balanced cell count culture in a model system.

61. The method according to claim 60, wherein the model system is an in-vitro model system, an ex-vivo model system, or an in-vivo model system.

62. A balanced cell count culture prepared by the method of any one of claims 33-61.

63. The balanced cell count culture of claim 62, wherein no less than 500 viable cells of each cell type of the two or more different cell types are present in the sample at the end of step (c) wherein the time period of step (c) is between 24 hours to 45 days.

64. The balanced cell count culture of culture of claim 62 or 63, wherein the time period of step (c) is between 3 days to 20 days.

65. The balanced cell count culture of any one of claims 62-64, wherein the different cell types comprise cells with cancer mutations, cancer cells from one or more subjects, primary cells from one or more subjects, cells from an organ system, cells from a disease model, cells from a variety of cell lines or any combination thereof.

66. The balanced cell count culture prepared by the method according to any one of claims 33-61, wherein the balanced cell count culture comprises two or more different cell types, wherein each of the two or more different cell types is represented with at least 1×10³ viable cells in the culture and wherein at least two of the cell types are sourced from different subjects.

67. The balanced cell count culture prepared by the method according to any one of claims 33-61, wherein the balanced cell count culture comprises two or more different cell types, wherein each of the two or more different cell types is represented with at least 1×10³ cells in the culture and wherein at least three of the cell types are derived from different cancer tissues.

68. The balanced cell count culture prepared by the method according to any one of claims 33-61, wherein the balanced cell count culture comprises two or more different cell types, wherein each of the two or more different cell types is represented with at least 1×10³ cells in the culture and wherein at least two of the cell types include cancer mutations that are different from each other.

69. The balanced cell count culture prepared by the method according to any one of claims 33-61, wherein the balanced cell count culture is implanted in an in-vitro, an ex-vivo, or an in-vivo model system.

70. The balanced cell count culture prepared by the method according to any one of claims 33-61, wherein no cell type of the balanced cell count culture outnumbers other cell types by 2 orders of magnitude or more.

71. The balanced cell count culture prepared by the method according to any one of claims 33-61, wherein the total number of each cell type of the two or more different cell types is within 2 orders of magnitude of each other.

72. A method of evaluating the impact of a candidate agent against two or more cell types, the method comprising;
i) preparing a balanced cell count culture of any one of claim 22-32 or 62-71;
ii) implanting the balanced cell count culture in a model system;
iii) treating the model system with a candidate agent over a duration of time; and
iv) evaluating the balanced cell count culture at the end of the duration of the time to determine phenotypic, genetic, and transcriptomic impact of the candidate agent on individual cells of the balanced cell count culture.

73. The method according to claim 72, wherein the model system is an in-vitro model system, an in-vivo model system, or an ex-vivo model system.

74. A method of creating a mosaic tumor comprising at least two or more different cell types in an in-vivo model system, the method comprising:
(i) preparing a balanced cell count culture of any one of claim 22-32 or 62-71, wherein at least two of the cell types are derived from different cancer tissues; and
(ii) implanting the balanced cell count culture in an in-vivo system, wherein the in-vivo system is a mammalian animal.

75. A method of evaluating the therapeutic efficacy of a candidate agent against individual cells of a mosaic tumor comprising at least two or more different cell types of claim 74, the method comprising,
(i) treating the mosaic tumor with the candidate agent over a duration of time;
(ii) dissociating cells of the treated mosaic tumor into individual cells;
(iii) evaluating the individual cells to determine phenotypic, genetic and transcriptomic expression of the individual cells of the mosaic tumor at the end of the duration of the time; and
(iv) determining the therapeutic efficacy of the candidate agent by comparing the phenotypic, genomic and transcriptomic expression of the individual cells of the mosaic tumor with phenotypic, genomic and transcriptomic expression of individual cells of an identical mosaic tumor that is not treated with the candidate agent.

76. A method of evaluating the impact of a candidate agent simultaneously against multiple cell types in an in-vivo system; the method comprising:
(i) preparing a balanced cell count culture of any one of claim 22-32 or 62-71, wherein at least two of the cell types are different from each other;
(ii) implanting the balanced cell count culture in an in-vivo system, wherein the in-vivo system is a mammalian animal;
(iii) treating the in-vivo system with a candidate agent over a duration of time;
(iv) evaluating the individual cells to determine phenotypic, genetic and transcriptomic expression of the individual cells of each of the multiple cell types at the end of the duration of the time; and
(v) determining impact of the candidate agent by comparing the phenotypic, genomic and transcriptomic expression of the individual cells of each of the multiple cell types in the in-vivo system with the phenotypic, genomic and transcriptomic expression of individual cells of each of multiple cell types of an identical in-vivo system that is not treated with the candidate agent.

77. A method of evaluating the impact of a candidate agent simultaneously against multiple cell types in an ex-vivo system; the method comprising:
(i) preparing a balanced cell count culture of any one of claim 22-32 or 62-71, wherein at least two of the cell types are different from each other;
(ii) implanting the balanced cell count culture in an ex-vivo system, wherein the ex-vivo system is an organoid;
(iii) treating the ex-vivo system with a candidate agent over a duration of time;
(iv) evaluating the individual cells to determine phenotypic, genetic and transcriptomic expression of the individual cells of each of the multiple cell types in the ex-vivo system at the end of the duration of the time; and
(v) determining impact of the candidate agent by comparing the phenotypic, genomic and transcriptomic expression of the individual cells of each of the multiple cell types in the ex-vivo system with the phenotypic, genomic and transcriptomic expression of individual cells of each of multiple cell types of an identical ex-vivo system that is not treated with the candidate agent.

78. A method of evaluating the impact of a candidate agent simultaneously against multiple cell types in an in-vitro system; the method comprising:
(i) preparing a balanced cell count culture of any one of claim 22-32 or 62-71, wherein at least two of the cell types are different from each other;
(ii) implanting the balanced cell count culture in an in-vitro system, wherein the in-vitro system is a 2D or a 3D in-vitro model system;
(iii) treating the in-vitro system with a candidate agent over a duration of time;
(iv) evaluating the individual cells to determine phenotypic, genetic and transcriptomic expression of the individual cells of each of the multiple cell types in the in-vitro system at the end of the duration of the time; and
(v) determining impact of the candidate agent by comparing the phenotypic, genomic and transcriptomic expression of the individual cells of each of the multiple cell types in the in-vitro system with the phenotypic, genomic and transcriptomic expression of individual cells of each of multiple cell types of an identical in-vitro system that is not treated with the candidate agent.

79. The method according to any one of claims 72-78, wherein the method comprises evaluating the impact of more than one candidate agent in a combination therapy.

80. The method according to claim 79, wherein the method comprises evaluating the impact of a combination of a first candidate agent and a second candidate agent.

81. The method according to claim 79 or 80 wherein the method evaluates drug synergy of the first candidate agent and the second candidate agent.

82. The method according to claim 80 or 81, wherein the method evaluates efficacy of the combination of the first candidate agent and the second candidate agent in suppressing a tumor growth.

83. A method of identifying a subject sub-population sensitive to a candidate agent, the method comprising:
(i) creating a balanced cell count culture of any one of claim 22-32 or 62-71 comprising multiple cell types, wherein the multiple cell types comprise cells from at least two different subjects;
(ii) implanting the balanced cell count culture in a model system;
(iii) treating the model system with a candidate agent over a duration of time;
(iv) evaluating the balanced cell count culture at the end of the duration of the time to determine phenotypic, genetic and transcriptomic impact of the candidate agent on individual cells of the balanced cell count culture; and
(v) identifying the subject sub-population sensitive to the candidate agent based on the evaluation of step iv).

84. A method of identifying a candidate agent target in a biological pathway, the method comprising:
(i) preparing a balanced cell count culture of any one of claim 22-32 or 62-71;
(ii) implanting the balanced cell count culture in a model system;
(iii) treating the model system with a candidate agent over a duration of time;
(iv) evaluating the balanced cell count culture at the end of the duration of the time to determine phenotypic, genetic, and transcriptomic impact of the candidate agent on individual cells of the balanced cell count culture; and
(v) identifying the candidate agent target in the biological pathway based on the evaluation of step iv).

85. A method of identifying therapeutic efficacy of a candidate agent for treating a disease, the method comprising:
(i) preparing a balanced cell count culture of any one of claims 32-56 wherein the different cell types comprise cells from two or more subjects having a disease;
(ii) implanting the balanced cell count culture in a model system;
(iii) treating the model system with a candidate agent over a duration of time;
(iv) evaluating the balanced cell count culture at the end of the duration of the time to determine phenotypic, genetic and transcriptomic impact of the candidate agent on individual cells of the balanced cell count culture; and
(v) determining the therapeutic efficacy of the candidate agent for treating the disease based on the evaluation of step iv).

86. The method according to any one of claim 72-73 or 75-85, wherein evaluating the resulting balanced cell count culture to determine genetic and transcriptomic impact of the candidate agent on individual cells of the balanced cell count culture comprises:
(i) dissociating cells of the resulting balanced cell count culture into single cells;
(ii) counting the number of cells of each of two or more different cell types remaining the resulting balanced cell count culture;
(iii) determining candidate agent-induced one or more gene expression changes for each cell type;
(iv) assigning a weighted score for each gene based on its predicted relevance to candidate agent sensitivity based on the calculated candidate agent-induced gene expression changes for each individual cells; and
(v) determining phenotypic, genetic and transcriptomic impact of the candidate agent on individual cells of the balanced cell count culture based on the weighted score of step iv).

87. The method according to any one of claim 72-73 or 75-86, wherein the candidate agent is, an agent that causes a therapeutic perturbation.

88. The method according to any one of claim 72-73 or 75-87, wherein the candidate agent is an agent selected from a small molecule, an antibody, a peptide, a gene editor, or a nucleic acid aptamer.

89. The method according to any one of claim 72-73 or 79-88, wherein determining the phenotypic, genetic, and transcriptomic impact comprises assessing the effect of the candidate agent on individual cells of the balanced cell count culture of any one of claim 22-31 or 61-69, or against a balanced cell count culture prepared by the method of any one of claims 32-60.

90. The method according to claim 89, wherein the effect of the candidate agent on individual cells of the balanced cell count culture is assessed by calculating gene expression for individual cells of the balanced cell count culture treated by the candidate agent and compare the gene expression with the gene expression for individual cells of an identical balanced cell count culture that is not treated by the candidate agent.

91. The method according to claim 89 or 90, wherein the effect of the candidate agent on individual cells of the balanced cell count culture is assessed by determining transcriptomic expression for individual cells of the balanced cell count culture treated by the candidate agent and compare the transcriptomic expression with the gene expression for individual cells of an identical balanced cell count culture that is not treated by the candidate agent.

92. The method according to any one of claims 89-91, wherein the effect of the candidate agent on individual cells of the balanced cell count culture is assessed by counting the number of viable individual cells of each of the cell types of the two or more different cell types in the balanced cell count culture in the treated by the candidate agent and comparing the number of viable individual cells of each of the cell types of the two or more different cell types in an identical balanced cell count culture that is not treated by the candidate agent.

93. The method according to any one of claim 72-73 or 79-92, wherein the evaluating genetic impact comprises single cell RNA sequencing of cells in the balanced cell count culture at the end of the duration of the time.

94. The method according to any of one of claim 72-73 or 79-93, wherein evaluating phenotypic changes comprises counting the number of viable individual cells of each of the cell types of the two or more different cell types at the end of the duration of the time.

95. The method according to any of one of claim 72-73 or 79-94, wherein evaluating transcriptomic impact comprises determining single-cell transcriptome profiles of cells of in the balanced cell count culture at the end of the duration of the time.

96. The method according to any of one of claim 72-73 or 74-95, wherein the duration of time is from 6 hours to 45 days, from 12 hours to 30 days, from 24 hours to 20 days, or from 72 hours to 14 days.

97. The method according to any of one of claim 72-73 or 74-96, wherein the duration of time is at least 24 hours.

98. The method according to any of one of claim 72-73 or 74-97, wherein the duration of time is 14 days.
